(19) United States
(12) Patent Application Publication
(10) Pub. No.: US 2007/0031348 A1
(43) Pub. Date: Feb. 8, 2007

(54) Use of oxygen in cosmetic or dermatological preparations

(76) Inventors: Franz Staeb, Echem (DE); Heidi Riedel, Hamburg (DE); Andreas Bleckmann, Ahrensburg (DE); Thomas Blatt, Wedel (DE); Christopher Mummert, Bienenbuettel (DE); Ludger Kolbe, Dohren (DE)

Correspondence Address: GREENBLUM & BERNSTEIN, P.L.C., 1950 ROLAND CLARKE PLACE, RESTON, VA, 20191, US

(21) Appl. No.: 10/571,528

(22) PCT Filed: Sep. 3, 2004
(86) PCT No.: PCT/EP04/52042

§ 371 Date: Sep. 28, 2006

(30) Foreign Application Priority Data
Sep. 13, 2003 (DE) 103 42 449.0

(51) Int. Cl. A61K 8/00; A61K 33/00

(52) U.S. Cl. 424/47; 424/600

(57) ABSTRACT

Use of molecular oxygen in cosmetic or dermatological preparations for the prophylaxis and treatment of a flawed appearance of the skin and/or of the cutaneous appendages as can arise through extrinsic and intrinsic and/or age-related or UV-induced disturbances.

The present invention relates to the use of oxygen in cosmetic or dermatological preparations for the prophylaxis against and treatment of skin aging phenomena, such as, for example, lines and wrinkles, slackening of the skin and tissue, disturbances in skin regeneration, circulation disorders of the skin, age spots and the like. In addition, the present invention relates to the use of oxygen in cosmetic and dermatological preparations for strengthening the barrier function of the skin.

Cosmetic skin care is primarily understood as meaning that the natural function of the skin as a barrier against environmental influences (e.g. dirt, chemicals, microorganisms) and against the loss of substances intrinsic to the body (e.g. water, natural fats, electrolytes) is strengthened or restored.

Impairment of this function may lead to increased resorption of toxic or allergenic substances or to attack by microorganisms, leading to toxic or allergic skin reactions.

In cases of aged skin, for example, regenerative renewal takes place at a slower rate, where, in particular, the water-binding capacity of the horny layer deteriorates. It therefore becomes inflexible, dry and chapped (“physiologically” dry skin). Barrier damage is the result. The skin becomes susceptible to negative environmental influences, such as the invasion of microorganisms, toxins and allergens. This may even result in toxic or allergic skin reactions.

In the case of pathologically dry and sensitive skin, barrier damage is present a priori. Epidermal intercellular lipids become defective or are formed in an inadequate amount or composition. The consequence is increased permeability of the horny layer and inadequate protection of the skin against the loss of hygroscopic substances and water.

Chronological skin aging is caused, for example, by endogenous genetically determined factors. The following structural damage and functional disorders, which can also fall under the term “senile xerosis”, arise, for example, in the epidermis and dermis as a result of aging:

a) dryness, roughness and formation of dryness wrinkles,

b) itching and

c) reduced refatting by sebaceous glands (e.g. after washing).

Exogenous factors, such as UV light and chemical noxae, can have a cumulative effect and, for example, accelerate or supplement the endogenous aging processes. In the epidermis and dermis, for example, the following structural damage and functional disorders arise in the skin in particular as a result of exogenous factors; these are more far-reaching than the degree and quality of the damage in the case of chronological aging:

d) visible vascular dilation (telangiectases, couperosis);

e) flaccidity and formation of wrinkles;

f) local hyperpigmentation, hypopigmentation and abnormal pigmentation (e.g. age spots) and

g) increased susceptibility to mechanical stress (e.g. cracking).

The present invention relates in particular to products for the care of skin aged naturally, and to the treatment of the damage caused by photo-aging, in particular of the phenomena listed under a) to g).

Products for the care of aged skin are known per se. They comprise, for example, retinoids (vitamin A acid and/or derivatives thereof) or vitamin A and/or derivatives thereof. Their effect on structural damage is, however, limited. Furthermore, in product development there are considerable difficulties in stabilizing the active ingredients to an adequate extent against oxidative decay. The use of products comprising vitamin A acid, moreover, often causes severe erythematous skin irritations. Retinoids can therefore only be used in low concentrations.

In particular, the present invention relates to cosmetic preparations having effective protection against harmful oxidation processes in the skin.

Products for protecting against harmful oxidation processes in the skin are known per se. Products which are intended to increase the oxygen supply to the skin have also already been described in the prior art. However, the prior art was unable to point the way to the present invention.

WO 02/05754 describes externally applicable preparations which comprise an oxygen carrier which is incorporated in molecularly dispersed form in a lipoid emulsion, and to the use thereof for the external treatment or the prevention of oxygen deficiency states of the skin.

The patent application DE 43 25 071 discloses a preparation for promoting circulation. For the transportation of gases—such as oxygen or carbon dioxide—fluorocarbons are used here.

U.S. Pat. No. 5,851,544 describes skin care preparations which increase the oxygen level in the skin would be desirable since the oxygen content in the skin is lower than in other regions of the body and, moreover, decreases with increasing age. It is therefore an object of U.S. Pat. No. 5,851,544 to increase the oxygen content of the skin in the long-term and/or to overcome oxygen deficiency states in the skin.

It is common to all these prior art preparations that an increased supply of oxygen for permanently balancing a lack of oxygen should be explicitly achieved.

However, on the other hand, besides the abovementioned desired effects, long-term oxygen therapies can, as is known, also have considerable disadvantages and accelerate, for example, endogenous aging processes. In particular free radical formation, oxidation of cellular lead structures and the like are problematic.

Moreover, it is known that undesired oxidation processes can arise in human and animal skin. In the essay “Skin Diseases Associated with Oxidative Injury” in “Oxidative Stress in Dermatology”, p. 323 ff. (Marcel Decker Inc., New York, Basel, Hong Kong, Editors: Jujrgen Fuchs, Frankfurt, and Lester Packer, Berkeley/Calif.), such oxidative damage to the skin and its more likely causes are explained.

It was therefore an object of the present invention to avoid the disadvantages of the prior art and, in particular, to remedy the damage to the skin caused by environmental noxae permanently, with lasting effect and without the risk of side-effects, or to prevent them. In particular it was the aim to provide skin care preparations and preparations for the care of skin aged naturally and also for the treatment of secondary damage of photo-aging, in particular the phenomena listed under a) to g). In particular, the effect of the preparations should be physiological, rapid and long-lasting.

Surprisingly, it has been found, and herein lies the attainment of these objects, that the
use of oxygen in cosmetic or dermatological preparations for the prophylaxis and treatment of a flawed appearance of the skin and/or of the cutaneous appendages, as can arise through extrinsic and intrinsic and/or age-related or UV-induced disturbances,
   remedies the disadvantages of the prior art.

The use according to the invention of oxygen or cosmetic or topical dermatological preparations with an effective content of non-molecularly bonded free oxygen—e.g. in the form of the examples given—surprisingly leads to a considerable improvement in various regenerative, restorative and vitalizing processes in the skin and the cutaneous appendages (nails, hair). In particular, the use according to the invention has a positive effect on the following skin properties:
direct defense function,
stress resistance,
nutrient supply,
skin color and complexion,
skin suppleness,
elasticity
skin structure,
equilibrium state and moisture balance.

Through the use of oxygen or cosmetic or topical dermatological preparations with an effective content of oxygen within the meaning of the present invention, the skin is surprisingly better protected against aging processes, structural and/or oxidative damage than would be possible with preparations of the prior art. In addition, the use according to the invention favors the restoration of the healthy skin state in cases of an existing disturbance and damage to the normal skin properties.

For the purposes of the present invention, “flawed appearance of the skin” is understood in particular as meaning the following phenomena:
lines and/or wrinkles,
slackening of skin and/or tissue,
disorders in regeneration of the skin and the cutaneous appendages,
circulation disorders of the skin,
age spots, pigment disorders and so-called uneven skin tone,
sensitive skin,
itching,
stress sensitivity of the skin,
inflamed and sensitive skin conditions,
dry skin conditions,
atopic eczema,
psoriasis
irregular or increased pore size.

In addition, on the basis of the cutaneous appendages (hair, nails), the following (aging) phenomena (“flawed appearance of the hair”) in particular are positively influenced through the use according to the invention:
growth deceleration,
deformation,
splitting,
chapping,
premature loss,
change in the structure and color.

Surprisingly, it has been found that the essential basis of the present invention is the pulsed short-term topical application of molecular oxygen. Through a short-term pulse (max. 1 hour) an oxygen deficit cannot be overcome in the relatively long-term, meaning that the present invention differs fundamentally from the prior art. However, short-term, pulse- or bolus-like stimulation of the intracellular and extracellular oxygen content has a signal function for the cells of the skin in as much as various mechanisms are thereby excited, as a result of which the cells overall are activated with regard to cell physiology without the disadvantages associated with long-term oxygen therapy arising. A permanent balancing of oxygen deficiency states in the skin is not intended to be achieved according to the invention. For the purposes of the present invention, however, it is advantageous to repeat the pulse-like application of molecular oxygen regularly—e.g. 2-3 times daily.

A particular advantage of the present invention is that the molecular oxygen is not bonded to carrier structures. This means that a rapid increase and decrease in the level of the gas through the various layers of the skin is possible. Long residence times and the risks associated therewith of oxidative and degenerative processes, which can arise after just a few hours in an atmosphere with a high content of oxygen and consequently would run counter to the inventive thinking, are thus excluded. The data on which this invention is based clearly demonstrate the advantage of a pulsed application with pure molecular oxygen over a long-lasting oxygen supply or that via carrier molecules.

The short-term oxygen supply according to the invention which is facilitated using the application forms listed in the examples leads to an inductive increase in the regenerative capacity of the skin. The positive effect of a short-term, topical oxygen treatment is evident essentially through the elimination of degenerative, age-related cellular processes, as occur in the cutaneous aging process. The resulting effect can also be referred to as oxygen lifting (“air-lift(ing)”).

For the purposes of the present invention, it is particularly advantageous to apply the oxygen to the skin in the form of self-foaming, foam-like, post-foaming or foamable cosmetic and dermatological preparations.

For the purposes of the present invention, “self-foaming”, “foam-like”, “post-foaming” and “foamable” are understood as meaning preparations from which foams can in principle be prepared—whether during the preparation process, whether during use by the consumer or in another way—by introducing one or more gases. In such foams, the gas bubbles are present in (any) distributed form in one (or more) liquid phase(s), where the (foamed) preparations do not necessarily have to have the appearance of foam in macroscopic terms. (Foamed) cosmetic or dermatological preparations according to the invention (for the sake of simplicity also referred to below as foams) may, for example, be macroscopically visibly dispersed systems of gases dispersed in liquids. The foam character can, however, for example, be visible only under a (light) microscope. Moreover, foams according to the invention are—particularly when the gas bubbles are too small to be recognized under a light microscope—also recognizable from the sharp increase in volume of the system.

Such preparations can advantageously be in the form of emulsions and, for the purposes of the present invention, advantageously comprise an emulsifier system which consists of
A. at least one emulsifier A chosen from the group of completely neutralized, partially neutralized or unneutralized, branched and/or unbranched, saturated and/or unsaturated fatty acids with a chain length of from 10 to 40 carbon atoms,
B. at least one emulsifier B chosen from the group of polyethoxylated fatty acid esters with a chain length of from 10 to 40 carbon atoms and with a degree of ethoxylation of from 5 to 100 and
C. at least one coemulsifier C chosen from the group of saturated and/or unsaturated, branched and/or unbranched fatty alcohols with a chain length of from 10 to 40 carbon atoms.

The emulsifier or the emulsifiers A are preferably chosen from the group of fatty acids which are completely or partially neutralized with customary alkalis (such as, for example, sodium hydroxide and/or potassium hydroxide, sodium carbonate and/or potassium carbonate, and also mono- and/or triethanolamine). For example, stearic acid and stearates, isostearic acid and isostearates, palmitic acid and palmitates, and myristic acid and myristates are particularly advantageous.

The emulsifier or the emulsifiers B are preferably chosen from following group: PEG-9 stearate, PEG-8 distearate, PEG-20 stearate, PEG-8 stearate, PEG-8 oleate, PEG-25 glyceryl trioleate, PEG-40 sorbitan lanolate, PEG-15 glyceryl ricinoleate, PEG-20 glyceryl stearate, PEG-20 glyceryl isostearate, PEG-20 glyceryl oleate, PEG-20 stearate, PEG-20 methylglucose sesquistearate, PEG-30 glyceryl iso-stearate, PEG-20 glyceryl laurate, PEG-30 stearate, PEG-30 glyceryl stearate, PEG-40 stearate, PEG-30 glyceryl laurate, PEG-50 stearate, PEG-100 stearate, PEG-150 laurate. For example, polyethoxylated stearic esters are particularly advantageous.

According to the invention, the coemulsifer or the coemulsifiers C are preferably chosen from the following group: behenyl alcohol (C₂₂H₄₅OH), cetearyl alcohol [a mixture of cetyl alcohol (Cl₆H₃₃OH) and stearyl alcohol (C₁₈H₃₇OH)], lanolin alcohols (wool wax alcohols which constitute the unsaponifiable alcohol fraction of wool wax which is obtained following saponification). Cetyl and cetyl stearyl alcohol are particularly preferred.

For the purposes of the present invention, it is particularly advantageous if the preparations are free from glyceryl stearate.

According to the invention, it is also advantageous to choose the weight ratios of emulsifier A to emulsifier B to coemulsifier C (A:B:C) as a:b:c, where a, b and c, independently of one another, can be rational numbers from 1 to 5, preferably from 1 to 3. A weight ratio of about 1:1:1 is particularly preferred.

For the purposes of the present invention, it is advantageous to choose the total amount of the emulsifiers A and B and of the coemulsifier C from the range from 2 to 20% by weight, advantageously from 5 to 15% by weight, in particular from 7 to 13% by weight, in each case based on the total weight of the formulation.

Preparations which comprise the abovementioned emulsifier system are particularly advantageous for the purposes of the present invention since the oxygen is released from these onto the skin during a single application over a period of less than one hour.

The present invention therefore also provides a method for the prophylaxis and treatment of a flawed appearance of the skin and/or of the cutaneous appendages, as can arise through extrinsic and intrinsic and/or age-related and UV-induced disturbances, characterized in that a cosmetic or dermatological preparation comprising an emulsifier system which consists of
A. at least one emulsifier A chosen from the group of completely neutralized, partially neutralized or unneutralized, branched and/or unbranched, saturated and/or unsaturated fatty acids with a chain length of from 10 to 40 carbon atoms,
B. at least one emulsifier B chosen from the group of polyethoxylated fatty acid esters with a chain length of from 10 to 40 carbon atoms and with a degree of ethoxylation of from 5 to 100 and
C. at least one coemulsifier C chosen from the group of saturated and/or unsaturated, branched and/or unbranched fatty alcohols with a chain length of from 10 to 40 carbon atoms
   and 1 to 90% by volume—based on the total volume of the preparation—of molecular oxygen, is applied at least once daily to the skin and/or the scalp.

For the purposes of the present invention, it is also advantageous if the cosmetic or dermatological preparations comprising oxygen used according to the invention are present in the form of gels. In this case, for the purposes of the present invention, the preparations comprise one or more hydrocolloids and/or gel formers in a concentration of from 0.1 to 8% by weight, preferably from 0.2 to 6% by weight and very particularly preferably from 0.3 to 4% by weight, in each case based on the total weight of the preparation.

The present invention therefore also provides a
method for the prophylaxis and treatment of a flawed appearance of the skin and/or of the cutaneous appendages as can arise through extrinsic and intrinsic and/or age-related and UV-induced disturbances, characterized in that a cosmetic or dermatological preparation comprising—based on the total weight of the preparation—0.1 to 8% by weight of one or more hydrocolloids and/or gel former and 1 to 90% by volume—based on the total volume of the preparation—of molecular oxygen, is applied at least once daily to the skin and/or the scalp.

“Hydrocolloid” is the technical abbreviation for the per se more correct name “hydrophilic colloid”. Hydrocolloids, also called thickeners or gel formers, are macromolecules which have a largely linear configuration and have intermolecular forces of interaction which permit secondary and primary valence bonds between the individual molecules and thus the formation of a reticular structure. They are sometimes water-soluble natural or synthetic polymers which form gels or viscous solutions in aqueous systems. They increase the viscosity of water by either binding water molecules (hydration) or else by absorbing and encapsulating the water into their interwoven macromolecules, at the same time as restricting the mobility of the water. Such water-soluble polymers represent a large group of chemically very different natural and synthetic polymers whose common feature is their solubility in water or aqueous media. A prerequisite for this is that these polymers have a number of hydrophilic groups sufficient for solubility in water and are not too greatly crosslinked. The hydrophilic groups may be nonionic, anionic, or cationic in nature, for example as follows:

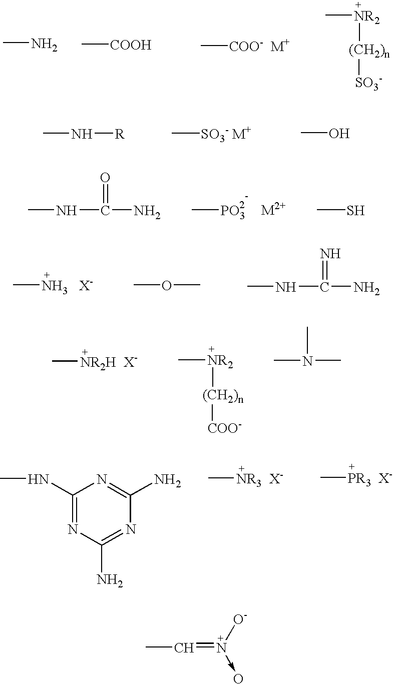

The group of cosmetically and dermatologically relevant hydrocolloids can be divided as follows into:

organic, natural compounds, such as, for example, agar agar, carrageen, tragacanth, gum arabic, alginates, pectins, polyoses, guar flour, carob bean flour, starch, dextrins, gelatin, casein, organic, modified natural substances, such as, for example carboxymethylcellulose and other cellulose ethers, hydroxyethylcellulose and hydroxypropylcellulose and the like, organic, completely synthetic compounds, such as, for example, polyacrylic and polymethacrylic compounds, vinyl polymers, polycarboxylic acids, polyethers, polyimines, polyamides, inorganic compounds, such as, for example, polysilicic acids, clay minerals, such as montmorillonites, zeolites, silicas.

Hydrocolloids which may be used advantageously according to the invention are agar agar, carrageen, tragacanth, gum arabic, alginates, pectins, polyoses, guar flour, carob bean flour, starch, dextrins, gelatin, casein, cellulose ethers, hydroxyethylcellulose and hydroxypropylcellulose derivatives, polyacrylic and polymethacrylic compounds, vinyl polymers, polycarboxylic acids, polyethers, polyimines, polyamides, polysilicic acids, clay minerals, zeolites, silicas.

Hydrocolloids preferred according to the invention are, for example, methylcelluloses, which is the term used to refer to the methyl ethers of cellulose. They are characterized by the following structural formula 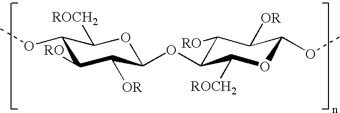
in which R may be a hydrogen or a methyl group.

Of particular advantage for the purposes of the present invention are the cellulose mixed ethers, which are generally likewise referred to as methylcelluloses and which, besides a dominant content of methyl groups, additionally contain 2-hydroxyethyl, 2-hydroxypropyl or 2-hydroxybutyl groups. Particular preference is given to (hydroxypropyl)methylcelluloses, for example those available under the trade name Methocel E4M from Dow Chemical Comp.

Also advantageous according to the invention is sodium carboxymethylcellulose, the sodium salt of the glycolic acid ether of cellulose for which R in structural formula I may be a hydrogen and/or CH₂—COONa. Particular preference is given to the sodium carboxymethylcellulose available under the trade name Natrosol Plus 330 CS from Aqualon, which is also referred to as cellulose gum.

For the purposes of the present invention, preference is also given to xanthan (CAS No. 11138-66-2), also called xanthan gum, which is an anionic heteropolysaccharide which is generally formed by fermentation from corn sugar and is isolated as the potassium salt. It is produced by Xanthomonas campestris and a few other species under aerobic conditions with a molecular weight of 2×10⁶ to 24×10⁶. Xanthan is formed from a chain with β-1,4-bonded glucose (cellulose) with side chains. The structure of the subgroups consists of glucose, mannose, glucuronic acid, acetate and pyruvate. Xanthan is the name for the first microbial anionic heteropolysaccharide. It is produced by Xanthomonas campestris and a few other species under aerobic conditions with a molecular weight of 2-15 10⁶. Xanthan is formed from a chain with β-1,4-bonded glucose (cellulose) with side chains. The structure of the subgroups consists of glucose, mannose, glucuronic acid, acetate and pyruvate. The number of pyruvate units determines the viscosity of the xanthan. Xanthan is produced in two-day batch cultures with a yield of 70-90%, based on carbohydrate used. Yields of 25-30 g/l are achieved here. After killing the culture, work-up takes place by precipitation with e.g. 2-propanol. Xanthan is then dried and ground.

An advantageous gel former for the purposes of the present invention is also carrageen, a gel-forming extract with a similar structure to that of agar from north Atlantic red algae which belong to the florideae (Chondrus crispus and Gigartina stellata).

The name carrageen is often used for the dried algae product and carrageenan for the extract from this. The carrageen precipitated from the hot-water extract of the algae is a colorless to sand-colored powder with a molecular weight range of 100 000-800 000 and a sulfate content of about 25%. Carrageen, which is very readily soluble in warm water; upon cooling forms a thixotropic gel, even if the water content is 95-98%. The strength of the gel is brought about by the double helix structure of the carrageen. In the case of carrageenan, a distinction is made between three main constituents: the gel-forming κ-fraction consists of D-galactose-4-sulfate and of 3,6-anhydro-α-D-galactose, which are alternately glycosidically bonded in the 1,3 and 1,4 position (in contrast, agar comprises 3,6-anhydro-α-L-galactose). The non-gelling λ-fraction is composed of 1,3-glycosidically bonded D-galactose-2-sulfate and 1,4-bonded D-galactose-2,6-disulfate radicals and is readily soluble in cold water. The ι-carrageenan formed from D-galactose4-sulfate in 1,3 bonding and 3,6-anhydro-α-D-galactose-2-sulfate in 1,4 bonding is both water-soluble and also gel-forming. Other carrageen types are likewise referred to with Greek letters: α, β, γ, μ, ν, ξ, π, ω, χ. The type of cations present (K⁺, NH₄⁺, Na⁺, Mg²⁺, Ca²⁺) also influences the solubility of the carrageens.

Polyacrylates are gelling agents likewise to be used advantageously for the purposes of the present invention. Polyacrylates advantageous according to the invention are acrylate-alkyl acrylate copolymers, in particular those chosen from the group of so-called carbomers or carbopols (Carbopol® is actually a registered trade mark of NOVEON Inc.). In particular, the acrylate-alkyl acrylate copolymers advantageous according to the invention are characterized by the following structure: 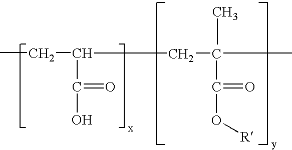
where R′ is a long-chain alkyl radical and x and y represent numbers which symbolize the respective stoichiometric proportion of the particular comonomers.

According to the invention, preference is given to acrylate copolymers and/or acrylate-alkyl acrylate copolymers which are available under the trade names Carbopol® 1382, Carbopol® 981 and Carbopol® 5984, Aqua SF-1 from NOVEON Inc. and as Aculyn® 33 from International Specialty Products Corp.

Also advantageous are copolymers of C10-30-alkyl acrylates and one or more monomers of acrylic acid, of methacrylic acid or esters thereof, which are crosslinked with an allyl ether of sucrose or an allyl ether of pentaerythritol.

Compounds which bear the INCI name “Acrylates/C 10-30 Alkyl Acrylate Crosspolymer” are advantageous. Particularly advantageous are those polymers available under the trade names Pemulen TR1 and Pemulen TR2 from NOVEON Inc.

Also advantageous are compounds which the INCI name “acrylates/C12-24 pareth-25 acrylate copolymer” (available under the trade name Synthalen® W2000 from 3V Inc.), which the INCI name “acrylates/steareth-20 methacrylate copolymer” (available under the trade name Aculyn® 22 from International Specialty Products Corp.), which the INCI name “acrylates/steareth-20 itaconate copolymer” (available under the trade name Structure 2001® from National Starch), which the INCI name “acrylates/aminoacrylates/C10-30 alkyl PEG-20 itaconate copolymer” (available under the trade name Structure Plus® from National Starch) and similar polymers.

The hydrocolloids particularly preferred according to the invention are: Acrylates Copolymer (AQUA SF-1), Acrylates/C 10-30 Alkyl Acrylate Crosspolymer (Carbopol ETD 2020), Xanthan Gum (Kelter).

The cosmetic or dermatological compositions according to the invention can advantageously be in the form of foamable preparations which, for example, are removed from aerosol containers and foamed in the process. Aerosol containers advantageous according to the invention are spray devices with a filling of the liquid or slurry-like substances which are under the pressure of a propellant (pressurized-gas or aerosol packagings). Containers of this type can be equipped with valves of very diverse construction which allow the contents to be removed as foam.

Foamable preparations according to the present invention which comprise the abovementioned emulsifier system can also advantageously be removed from pump atomizers (pump dispensers) which are free from propellant gas and are operated mechanically. Of particular advantage for the purposes of the present invention are pump systems which operate without compressed gas, but with a filter which brings about specific swirling movements.

Suitable pressurized gas containers for the purposes of the present invention are primarily cylindrical vessels made of metal (aluminum, tinplate, contents <1000 ml), protected or shatter-resistant glass or plastic (contents <220 ml) or shattering glass or plastic (contents <150 ml), in the choice of which compressive strength and breaking strength, corrosion resistance, ease of filling, or ease of sterilizing etc., but also esthetic aspects, handleability, printing properties etc. play a role. The maximum permissible operating pressure of spray cans made of metal at 50° C. is 12 bar and the maximum fill volume at this temperature is about 90% of the total volume. For glass and plastic cans, the values for the operating pressure are lower and dependent on the size of the container and the propellant (whether liquefied, compressed or dissolved gas).

For the purposes of the present invention, cans made of tinplate, aluminum and glass are particularly advantageous. For reasons of corrosion protection, metal cans can be coated on the inside (silver- or gold-coated), for which purpose all standard commercial internal protective coatings are suitable. For the purposes of the present invention, preference is given to polyester, epoxyphenol and polyamide-imide coatings. Film laminates made of polyethylene (PE), polypropylene (PP) and/or polyethylene terephthalate (PET) on the inside of the cans are also advantageous, in particular for cans made of tinplate.

The pressurized gas containers are usually single-part or two-part, but in most cases three-part cylindrical, conical or differently shaped. If plastics are used as the spray container material, then these should be resistant to chemicals and the sterilization temperature, gas-tight, impact-resistant and stable to internal pressures in excess of 12 bar. In principle, polyacetals and polyamides are suitable for spray container purposes.

The internal construction of the spray cans and the valve construction are very varied, depending on the intended use and the physical nature of the ingredient—e.g. whether it is in the form of a two-phase or three-phase system—and can be determined by the person skilled in the art by simple exploratory investigations without inventive activity. For suitable variants, reference may be made to the “Aerosol Technologie Handbuch der Aerosol-Verpackung” [Aerosol Technology Handbook of Aerosol Packaging] (Wolfgang Tauscher, Melcher Verdag GmbH Heidelberg/Munich, 1996).

Valves which are advantageous according to the invention can be designed with or without riser tube. The individual components from which valves according to the invention are usually constructed preferably consist of the following materials:
Disk: tinplate: uncoated, gold- or clear-coated, film-laminated (PE, PP or PET) aluminum: uncoated, silver- or gold-coated, different coating variants, Stoner-Mudge design
Seal: natural or synthetic elastomers or thermoplastic (sleeve gaskets, film-lined made of PE or PP) internal and external seals, e.g. made of perbunan, buna, neoprene, butyl, CLB, LDPE, viton, EPDM, chlorobutyl, bromobutyl and/or diverse compounds
Cone: PA, POM, brass and diverse special materials, standard bores (e.g.: 0.25 to 0.70 mm or 2×0.45 to 2 x 1.00 mm), various shaft diameters
Spring: metal, particularly preferably V2A, stainless steel; plastic and also elastomer
Casing: standard and impact VPH bores, RPT bores or slit for upside-down applications materials: e.g. polyacetal, PA, PE, POM and the like
Riser tube: plastic (polymer resin), e.g. PE, PP, PA or polycarbonate

Advantageous spray heads for the purposes of the present invention are, for example, foaming heads for upright use (hold can vertically) or foaming heads for upside-down application using one or more channels.

For the purposes of the present invention, the volume content of propellant gas is chosen from the range from 0.1 to 30% by volume, based on the total volume of fill material and propellant gas (corresponding to a volume content of 70 to 99.9% by volume of fill material).

A particularly preferred propellant gas for the purposes of the present invention is oxygen, although air or other oxygen-containing gas mixtures are also suitable in principle.

It is advantageous for the purposes of the present invention to choose the total amount of oxygen in the foamed preparations for the purposes of the present invention from the range from 0.5 to 90% by volume, advantageously from 5 to 50% by volume, in particular from 10 to 40% by volume, in each case based on the total volume of the formulation.

The cosmetic or dermatological formulations for the purposes of the present invention may have the customary composition and be used for cosmetic or dermatological light protection, and also for the treatment, care and cleansing of the skin, of the lips and of skin appendages (nails and/or hair) and as make-up product in decorative cosmetics.

Just as emulsions with liquid and solid consistency are used as cosmetic cleansing lotions or cleansing creams, the preparations for the purposes of the present invention can also constitute “cleansing foams”, which can be used, for example, for removing make-up or as mild washing and showering foam—if appropriate also for damaged skin. Such cleansing foams may advantageously also be used as so-called rinse off preparations which are rinsed off following application to the skin.

The cosmetic and/or dermatological preparations according to the invention can also advantageously be in the form of a care foam for the hair and/or the scalp, in particular a foam for arranging the hair, a foam which is used while blow-drying the hair, a styling and treatment foam.

Depending on their formulation, cosmetic or topical dermatological compositions for the purposes of the present invention can, for example, be used as skin protection cream, cleansing milk, day or night cream etc. It is in some cases possible and advantageous to use the compositions according to the invention as a base for pharmaceutical formulations.

For use, the cosmetic and dermatological preparations are applied to the skin and/or the hair in an adequate amount in the manner customary for cosmetics.

The cosmetic and dermatological preparations according to the invention can comprise cosmetic auxiliaries as are customarily used in such preparations, e.g. preservatives, preservative aids, complexing agents, bactericides, perfumes, substances for increasing foaming, dyes, pigments which have a coloring action, thickeners, moisturizing and/or humectant substances, fillers which improve the feel on the skin, fats, oils, waxes or other customary constituents of a cosmetic or dermatological formulation, such as alcohols, polyols, polymers, foam stabilizers, electrolytes, organic solvents or silicone derivatives.

Advantageous preservatives for the purposes of the present invention are, for example, formaldehyde donors (such as, for example, DMDM hydantoin, which is available, for example, under the trade name Glydant™ from Lonza), mixtures containing iodopropyl butylcarbamate (e.g. those available under the trade names Glycacil-L, Glycacil-S from Lonza and/or Dekaben LMB from Jan Dekker), parabens (i.e. alkyl p-hydroxybenzoates, such as methyl-, ethyl-, propyl- and/or butylparaben), phenoxyethanol, ethanol, benzoic acid and the like. In addition, the preservative system according to the invention also usually advantageously comprises preservative aids, such as, for example, octoxyglycerol, glycine soya etc.

Advantageous complexing agents for the purposes of the present invention are, for example, EDTA, [S,S]-ethylenediamine disuccinate (EDDS), which is available, for example, under the trade name Octaquest from Octel, pentasodium ethylenediamine tetramethylenephosphonate, which is available, for example, under the trade name Dequest 2046 from Monsanto and/or iminodisuccinic acid, which is available, inter alia, from Bayer AG under the trade names Iminodisuccinate VP OC 370 (about 30% strength solution) and Baypure CX 100 solid.

Particularly advantageous preparations are also obtained when antioxidants are used as additives or active ingredients. According to the invention, the preparations advantageously comprise one or more antioxidants. Favorable, but nevertheless optional, antioxidants which may be used are all antioxidants customary or suitable for cosmetic and/or dermatological applications.

For the purposes of the present invention, water-soluble antioxidants can be particularly advantageously used, such as, for example, vitamins, e.g. ascorbic acid and derivatives thereof.

Preferred antioxidants are also vitamin E and derivatives thereof and vitamin A and derivatives thereof.

The amount of antioxidants (one or more compounds) in the preparations is preferably 0.001 to 30% by weight, particularly preferably 0.05 to 20% by weight, in particular 0.1 to 10% by weight, based on the total weight of the preparation.

If vitamin E and/or derivatives thereof are the antioxidant or the antioxidants, it is advantageous to choose their respective concentrations from the range from 0.001 to 10% by weight, based on the total weight of the formulation.

If vitamin A or vitamin A derivatives or carotenes or derivatives thereof are the antioxidant or the antioxidants, it is advantageous to choose their respective concentrations from the range from 0.001 to 10% by weight, based on the total weight of the formulation.

It is particularly advantageous when the cosmetic preparations according to the present invention comprise cosmetic or dermatological active ingredients, preferred active ingredients being antioxidants which can protect the skin against oxidative stress.

Further advantageous active ingredients for the purposes of the present invention are natural active ingredients and/or derivatives thereof, such as, for example, alpha-lipoic acid, phytoene, D-biotin, coenzyme Q10, alpha-glucosylrutin, carnitine, carnosine, natural and/or synthetic isoflavonoids, creatine, creatinine, taurine and/or β-alanine, and 8-hexadecene-1,16-dicarboxylic acid (dioic acid, CAS number 20701-68-2; provisional INCI name Octadecenedioic acid).

Formulations according to the invention which comprise, for example, known antiwrinkle active ingredients, such as flavone glycosides (in particular α-glucosylrutin), coenzyme Q10, vitamin E and/or derivatives and the like are particularly advantageously suitable for the prophylaxis and treatment of cosmetic or dermatological changes in the skin, as arise, for example, during skin ageing (such as, for example, dryness, roughness and formation of dryness wrinkles, itching, reduced refatting (e.g. after washing), visible vascular dilations (teleangiectasis, couperosis), flaccidity and formation of wrinkles and lines, local hyperpigmentation, hypopigmentation and incorrect pigmentation (e.g. age spots), increased susceptibility to mechanical stress (e.g. cracking) and the like). In addition, they are advantageously suitable to counter the appearance of dry or rough skin.

It is particularly advantageous for the purposes of the present invention when the further active ingredients are present in encapsulated form such that they are physically separate from the formulation constituents (or further, non-compatible active ingredients) and/or from the potentially oxidizing oxygen. In this connection—depending on the nature of the active ingredient used—permanent encapsulations, i.e. capsules from which the active ingredients are not released into the cosmetic preparation or the skin (advantageously, for example, for UV filter substances) or non-permanent encapsulations are conceivable.

Advantageous encapsulations consist, for example, of plastics. It is further advantageous to encapsulate the further active ingredients into collagen matrices and other customary encapsulation materials, e.g. as cellulose encapsulations, in gelatin, wax matrices or liposomally encapsulated. In particular, wax matrices, as described in DE-A 43 08 282 have proven to be favourable. Particularly advantageous encapsulation forms for the purposes of the present invention are also cyclodextrin complexes of the other active ingredients.

Also advantageous are, for example, encapsulations which are obtainable by sol gel microtechnology. Here, the active ingredients are enclosed in an inert silica membrane, ultimately thus encapsulated in glass beads. For the purposes of the present invention, encapsulated active ingredients may advantageously also be used in the form of aqueous dispersions.

Encapsulated active ingredients are suitable in particular for the preparation of particularly skin compatible (sensitive) products. Moreover, it is of course advantageous to use active ingredients with potential skin irritancy in encapsulated form.

In addition, it is also advantageous for the purposes of the present invention to use active ingredient capsules referred to as “microbeads”. Advantageous “microbeads” are, for example, those listed below:

| Trade name | Manufacturer | Composition (INCI) |
|---|---|---|
| Unispheres | Induchem | Lactose + Cellulose + Hydroxypropyl |
| UEL-611 | | Methylcellulose + CI 77707 + Tocopherol Acetate |
| Unispheres | Induchem | Lactose + Cellulose + Hydroxypropyl |
| RP-572 | | Methylcellulose + Panthenyl Triacetate + CI |
| | | 7360 |
| Unispheres | Induchem | Lactose + Cellulose + Hydroxypropyl |
| UT-513 | | Methylcellulose + CI 77707 + Tocopherol |
| Macrobeads | Wiblosan | Cetearyl Alcohol + Acrylates Copolymer + Paraffinum |
| | | Liquidum + Microcrystalline |
| | | Cellulose + Bisabolol + Tocopherol Acetate + CI 74260 |

The water phase of the preparations according to the present invention can advantageously comprise customary cosmetic auxiliaries, such as, for example, alcohols, in particular those of low carbon number, preferably ethanol and/or isopropanol, diols or polyols of low carbon number, and ethers thereof, preferably propylene glycol, glycerol, butylene glycol, ethylene glycol, ethylene glycol monoethyl or monobutyl ether, propylene glycol monomethyl, monoethyl or monobutyl ether, diethylene glycol monomethyl or monoethyl ether and analogous products, polymers, foam stabilizers, electrolytes, and in particular one or more thickeners which can be chosen advantageously from the group consisting of silicon dioxide, aluminum silicates, polysaccharides or derivatives thereof, e.g. hyaluronic acid, xanthan gum, hydroxypropylmethylcellulose, particularly advantageously from the group of polyacrylates, preferably a polyacrylate from the group of so-called carbopols [from Bf. Goodrich], for example, carbopol grades 980, 981, 1382, 2984, 5984, ETD 2020, ETD 2050, Ultrez 10, in each case individually or in combination.

In addition, the preparations according to the invention can advantageously also comprise self-tanning substances, such as, for example, dihydroxyacetone and/or melanin derivatives in concentrations of from 1% by weight to 8% by weight, based on the total weight of the preparation.

In addition, the preparations according to the present invention can advantageously also comprise repellants for protection against flies, ticks and spiders and the like. For example, N,N-diethyl-3-methylbenzamide (trade name: Metadelphene, “DEET”), dimethyl phthalate (trade name; Palatinol M, DMP), and in particular ethyl 3-(N-n-butyl-N-acetylamino)propionate (available under the trade name lnsekt Repellent® 3535 from Merck) are advantageous. The repellents can either be used individually or in combination.

Moisturizers is the term used to refer to substances or mixtures of substances which impart to cosmetic or dermatological preparations the property, following application or distribution on the surface of the skin, of reducing moisture release by the horny layer (also called transepidermal water loss (TEWL)) and/or of positively influencing hydration of the horny layer.

Advantageous moisturizers for the purposes of the present invention are, for example, glycerol, lactic acid and/or lactates, in particular sodium lactate, butylene glycol, propylene glycol, biosaccharide gum-1, glycine soya, ethylhexyloxyglycerol, pyrrolidonecarboxylic acid and uric acid. In addition, it is particularly advantageous to use polymeric moisturizers from the group of water-soluble and/or water-swellable and/or water-gellable polysaccharides. Hyaluronic acid, chitosan and/or a fucose-rich polysaccharide, which is filed in the Chemical Abstracts under the registry number 178463-23-5 and which is available, for example, under the name Fucogel® 1000 from SOLABIA S.A., for example, are particularly advantageous. Moisturizers can advantageously also be used as antiwrinkle active ingredients for the prophylaxis and treatment of cosmetic or dermatological changes in the skin, as arise, for example, during skin ageing.

The cosmetic or dermatological preparations according to the invention can also advantageously, but not necessarily, comprise fillers, which, for example, further improve the sensory and cosmetic properties of the formulations and, for example, bring about or enhance a velvety or silky feel on the skin. Advantageous fillers for the purposes of the present invention are starch and starch derivatives (such as, for example, tapioca starch, distarch phosphate, aluminum or sodium starch octenylsuccinate and the like), pigments which have neither a primarily UV filter effect nor a coloring effect (such as, for example, boron nitride etc.) and/or Aerosils® (CAS No. 7631-86-9).

The oil phase of the formulations according to the invention is advantageously chosen from the group of polar oils, for example from the group of lecithins and of fatty acid triglycerides, namely the triglycerol esters of saturated and/or unsaturated, branched and/or unbranched alkanecarboxylic acids with a chain length of from 8 to 24, in particular 12 to 18, carbon atoms. The fatty acid triglycerides can, for example, be chosen advantageously from the group of synthetic or semisynthetic and natural oils, such as, for example, cocoglyceride, olive oil, sunflower oil, soya oil, peanut oil, rapeseed oil, almond oil, palm oil, coconut oil, castor oil, wheatgerm oil, grapeseed oil, thistle oil, evening primrose oil, macadamia nut oil and the like.

Also advantageous according to the invention are, for example, natural waxes of animal and vegetable origin, such as, for example, beeswax and other insect waxes, and berry wax, shea butter and/or lanolin (wool wax).

For the purposes of the present invention, further advantageous polar oil components can also be chosen from the group of esters of saturated and/or unsaturated, branched and/or unbranched alkanecarboxylic acids with a chain length of from 3 to 30 carbon atoms and saturated and/or unsaturated, branched and/or unbranched alcohols with a chain length of from 3 to 30 carbon atoms, and from the group of esters of aromatic carboxylic acids and saturated and/or unsaturated, branched and/or unbranched alcohols with a chain length of from 3 to 30 carbon atoms. Such ester oils can then advantageously be chosen from the group consisting of octyl palmitate, octyl cocoate, octyl isostearate, octyl dodecyl myristate, octyldodecanol, cetearyl isononanoate, isopropyl myristate, isopropyl palmitate, isopropyl stearate, isopropyl oleate, n-butyl stearate, n-hexyl laurate, n-decyl oleate, isooctyl stearate, isononyl stearate, isononyl isononanoate, 2-ethylhexyl palmitate, 2-ethylhexyl laurate, 2-hexyldecyl stearate, 2-octyldodecyl palmitate, stearyl heptanoate, oleyl oleate, oleyl erucate, erucyl oleate, erucyl erucate, tridecyl stearate, tridecyl trimellitate, and synthetic, semisynthetic and natural mixtures of such esters, such as, for example, jojoba oil.

The oil phase can also advantageously be chosen from the group of dialkyl ethers and dialkyl carbonates, advantageous examples being dicaprylyl ether (Cetiol OE) and/or dicaprylyl carbonate, for example that available under the trade name Cetiol CC from Cognis.

It is also preferred to select the oil component or the oil components from the group consisting of isoeicosane, neopentyl glycol diheptanoate, propylene glycol dicaprylate/dicaprate, caprylic/capric/diglyceryl succinate, butylene glycol dicaprylate/dicaprate, C₁₂₋₁₃-alkyl lactate, di-C₁₂₋₁₃-alkyl tartrate, triisostearin, dipentaerythrityl hexacaprylate/hexacaprate, propylene glycol monoisostearate, tricaprylin, dimethylisosorbide. It is particularly advantageous if the oil phase of the formulations according to the invention has a content of C₁₂₋₁₅-alkyl benzoate, or consists entirely of this.

Advantageous oil components are also, for example, butyloctyl salicylate (for example that available under the trade name Hallbrite BHB from CP Hall), hexadecyl benzoate and butyloctyl benzoate and mixtures thereof (Hallstar AB) and/or diethylhexyl naphthalate (Hallbrite TQ or Corapan TQ from H&R).

Any desired mixtures of such oil and wax components can also be used advantageously for the purposes of the present invention.

In addition, the oil phase can likewise advantageously also comprise non-polar oils, for example those which are chosen from the group of branched and unbranched hydrocarbons and hydrocarbon waxes, in particular mineral oil, vaseline (petrolatum), paraffin oil, squalane and squalene, polyolefins, hydrogenated polyisobutenes and isohexadecane. Among the polyolefins, polydecenes are the preferred substances.

The oil phase can also advantageously have a content of cyclic or linear silicone oils or consist entirely of such oils, although it is preferred to use an additional content of other oil phase components apart from the silicone oil or the silicone oils.

Silicone oils are high molecular weight synthetic polymeric compounds in which silicon atoms are joined in a chain-like and/or reticular manner via oxygen atoms and the remaining valences of the silicon are saturated by hydrocarbon radicals (in most cases methyl, less often ethyl, propyl, phenyl groups etc.). Systematically, the silicone oils are referred to as polyorganosiloxanes. The methyl-substituted polyorganosiloxanes, which are the most important compounds of this group in terms of amount and are characterized by the following structural formula 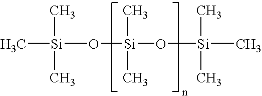
are also referred to as polydimethylsiloxane or Dimethicone (INCI). Dimethicones have various chain lengths and various molecular weights.

Particularly advantageous polyorganosiloxanes for the purposes of the present invention are, for example, dimethylpolysiloxanes [poly(dimethylsiloxane)], which are available, for example, under the trade names Abil 10 to 10 000 from Th. Goldschmidt. Also advantageous are phenylmethylpolysiloxanes (INCI: Phenyl Dimethicone, Phenyl Trimethicone), cyclic silicones (octamethylcyclotetrasiloxane and decamethylcyclopentasiloxane), which are also referred to in accordance with INCI as Cyclomethicone, amino-modified silicones (INCI: Amodimethicone) and silicone waxes, e.g. polysiloxane-polyalkylene copolymers (INCI: Stearyl Dimethicone and Cetyl Dimethicone) and dialkoxydimethylpolysiloxanes (Stearoxy Dimethicone and Behenoxy Stearyl Dimethicone), which are available as various Abil wax grades from Th. Goldschmidt. However, other silicone oils can also be used advantageously for the purposes of the present invention, for example cetyldimethicone, hexamethylcyclotrisiloxane, polydimethylsiloxane, poly(methylphenylsiloxane).

The preparations according to the present invention can also advantageously comprise one or more substances from the following group of siloxane elastomers, for example in order to increase the water resistance and/or the light protection factor of the products:
(a) siloxane elastomers which contain the units R₂SiO and RSiO_{1.5} and/or R₃SiO_{0.5} and/or SiO₂,
   where the individual radicals R, in each case independently of one another, are hydrogen, C₁₋₂₄-alkyl (such as, for example, methyl, ethyl, propyl) or aryl (such as, for example, phenyl or tolyl), alkenyl (such as, for example, vinyl), and the weight ratio of the units R₂SiO to RSiO_{1.5} is chosen from the range from 1:1 to 30:1;
(b) siloxane elastomers which are insoluble and swellable in silicone oil and which are obtainable by the addition reaction of an organopolysiloxane (1) which contains silicon-bonded hydrogen with an organopolysiloxane (2) which contains unsaturated aliphatic groups,
   where the quantitative amounts used are chosen such that the amount of hydrogen in the organopolysiloxane (1) or in the unsaturated aliphatic groups of the organopolysiloxane (2)
   is in the range from 1 to 20 mol % when the organopolysiloxane is non-cyclic and
   is in the range from 1 to 50 mol % when the organopolysiloxane is cyclic.

For the purposes of the present invention, the siloxane elastomer or elastomers are advantageously present in the form of spherical powders or in the form of gels.

Siloxane elastomers present in the form of spherical powders which are advantageous according to the invention are those with the INCI name Dimethicone/Vinyl Dimethicone Crosspolymer, for example that available from DOW CORNING under the trade names DOW CORNING 9506 Powder.

It is particularly preferred when the siloxane elastomer is used in combination with oils from hydrocarbons of animal and/or vegetable origin, synthetic oils, synthetic esters, synthetic ethers or mixtures thereof.

It is very particularly preferred when the siloxane elastomer is used in combination with unbranched silicone oils which are liquid or pasty at room temperature or cyclic silicone oils or mixtures thereof. Organopolysiloxane elastomers with the INCI name Dimethicone/Polysilicone-11, very particularly the Gransil grades obtainable from Grant Industries Inc. GCM, GCM-5, DMG-6, CSE gel, PM-gel, LTX, ININ gel, AM-18 gel and/or DMCM-5, are particularly advantageous.

It is very extremely preferred when the siloxane elastomer is used in the form of a gel of siloxane elastomer and a lipid phase where the content of the siloxane elastomer in the gel is 1 to 80% by weight, preferably 0.1 to 60% by weight, in each case based on the total weight of the gel.

It is advantageous for the purposes of the present invention to choose the total amount of the siloxane elastomers (active content) from the range from 0.01 to 10% by weight, advantageously from 0.1 to 5% by weight, in each case based on the total weight of the formulation.

The cosmetic and dermatological preparations according to the invention can comprise dyes and/or color pigments, particularly when they are in the form of decorative cosmetics. The dyes and color pigments can be chosen from the corresponding positive list in the Cosmetics Directive or the EC list of cosmetic colorants. In most cases, they are identical to dyes approved for foods. Advantageous color pigments are, for example, titanium dioxide, mica, iron oxides (e.g. Fe₂O₃, Fe₃O₄, FeO(OH)) and/or tin oxide. Advantageous dyes are, for example, carmine, Prussian blue, chromium oxide green, ultramarine blue and/or manganese violet. It is particularly advantageous to choose the dyes and/or the color pigments from the Rowe Color Index, 3rd Edition, Society of Dyers and Colorists, Bradford, England, 1971.

If the formulations according to the invention are in the form of products which are used on the face, it is favorable to choose one or more substances from the following group as the dye: 2,4-dihydroxyazobenzene, 1-(2′-chloro-4′-nitro-1′-phenylazo)-2-hydroxynaphthalene, Ceres red, 2-(sulpho-1-naphthylazo)-1-naphthol-4-sulfonic acid, calcium salt of 2-hydroxy-1,2′-azonaphthalene-1′-sulfonic acid, calcium and barium salts of 1-(2-sulpho-4-methyl-1-phenylazo)-2-naphthylcarboxylic acid, calcium salt of 1-(2-sulpho-1-naphthylazo)-2-hydroxynaphthalene-3-carboxylic acid, aluminum salt of 1-(4-sulpho-1-phenylazo)-2-naphthol-6-sulfonic acid, aluminum salt of 1-(4-sulpho-1-naphthylazo)-2-naphthol-3,6-disulfonic acid, 1-(4-sulpho-1-naphthylazo)-2-naphthol-6,8-disulfonic acid, aluminum salt of 4-(4-sulpho-1-phenylazo)-1-(4-sulphophenyl)-5-hydroxypyrazolone-3-carboxylic acid, aluminum and zirconium salts of 4,5-dibromofluorescein, aluminum and zirconium salts of 2,4,5,7-tetrabromofluorescein, 3′,4′,5′,6′-tetrachloro-2,4,5,7-tetrabromofluorescein and its aluminum salt, aluminum salt of 2,4,5,7-tetraiodofluorescein, aluminum salt of quinophthalonedisulfonic acid, aluminum salt of indigodisulfonic acid, red and black iron oxide (CIN: 77 491 (red) and 77 499 (black)), iron oxide hydrate (CIN: 77 492), manganese ammonium diphosphate and titanium dioxide.

Also advantageous are oil-soluble natural dyes, such as, for example, paprika extracts, β-carotene or cochineal.

Also advantageous for the purposes of the present invention are formulations with a content of pearlescent pigments. Preference is given in particular to the types of pearlescent pigments listed below:
1. Natural pearlescent pigments, such as, for example,
   “pearl essence” (guanine/hypoxanthin mixed crystals from fish scales) and
   “mother-of-pearl” (ground mussel shells)
2. Monocrystalline pearlescent pigments, such as, for example, bismuth oxychloride (BiOCl)
3. Layer-substrate pigments: e.g. mica/metal oxide

Bases for pearlescent pigments are, for example, pulverulent pigments or castor oil dispersions of bismuth oxychloride and/or titanium dioxide, and bismuth oxychloride and/or titanium dioxide on mica. The lustre pigment listed under CIN 77163, for example, is particularly advantageous.

Also advantageous are, for example, the following types of pearlescent pigments based on mica/metal oxide:

| Group | Coating/layer thickness | Color |
|---|---|---|
| Silver-white pearlescent | TiO₂: 40-60 nm | Silver |
| pigments | | |
| Interference pigments | TiO₂: 60-80 nm | Yellow |
| | TiO₂: 80-100 nm | Red |
| | TiO₂: 100-140 nm | Blue |
| | TiO₂: 120-160 nm | Green |
| Color lustre pigments | Fe₂O₃ | Bronze |
| | Fe₂O₃ | Copper |
| | Fe₂O₃ | Red |
| | Fe₂O₃ | Red-violet |
| | Fe₂O₃ | Red-green |
| | Fe₂O₃ | Black |
| Combination pigments | TiO₂/Fe₂O₃ | Gold shades |
| | TiO₂/Cr₂O₃ | Green |
| | TiO₂/Prussian blue | Deep blue |
| | TiO₂/carmine | Red |

Particular preference is given, for example, to the pearlescent pigments obtainable from Merck under the trade names Timiron, Colorona or Dichrona.

The list of given pearlescent pigments is not of course intended to be limiting. Pearlescent pigments which are advantageous for the purposes of the present invention are obtainable by numerous methods known per se. For example, other substrates apart from mica can be coated with further metal oxides, such as, for example, silica and the like. SiO₂ particles coated with, for example, TiO₂ and Fe₂O₃ (“ronaspheres”), which are sold by Merck and are particularly suitable for the optical reduction of fine lines, are advantageous.

It can, moreover, be advantageous to dispense completely with a substrate such as mica. Particular preference is given to iron pearlescent pigments prepared without the use of mica. Such pigments are obtainable, for example, under the trade name Sicopearl Kupfer 1000 from BASF.

In addition, also particularly advantageous are effect pigments which are obtainable under the trade name Metasomes Standard/Glitter in various colors (yellow, red, green, blue) from Flora Tech. The glitter particles are present here in mixtures with various auxiliaries and dyes (such as, for example, the dyes with the Color Index (CI) numbers 19140, 77007, 77289, 77491).

The dyes and pigments may be present either individually or in a mixture, and can be mutually coated with one another, different coating thicknesses generally giving rise to different color effects. The total amount of dyes and color-imparting pigments is advantageously chosen from the range from, for example, 0.1% by weight to 30% by weight, preferably from 0.5 to 15% by weight, in particular from 1.0 to 10% by weight, in each case based on the total weight of the preparations.

For the purposes of the present invention, it is also advantageous to provide cosmetic and dermatological preparations whose main purpose is not protection against sunlight, but which nevertheless have a content of further UV protection substances. Thus, for example, UV-A and/or UV-B filter substances are usually incorporated into daycreams or make-up products. UV protection substances, like antioxidants and, if desired, preservatives, also constitute effective protection of the preparations themselves against spoilage. Also favourable are cosmetic and dermatological preparations in the form of a sunscreen.

Accordingly, for the purposes of the present invention, the preparations preferably comprise at least one further UV-A, UV-B and/or broadband filter substance. The formulations can, but do not necessarily, optionally also comprise one or more organic and/or inorganic pigments as UV filter substances, which may be present in the water phase and/or the oil phase.

In addition, the preparations according to the present invention can also advantageously be in the form of so-called oil-free cosmetic or dermatological emulsions, which comprise a water phase and at least one UV filter substance which is liquid at room temperature as a further phase, and which may particularly advantageously also be free from further oil components.

For the purposes of the present invention, particularly advantageous UV filter substances which are liquid at room temperature are homomenthyl salicylate (INCI: Homosalate), 2-ethylhexyl 2-cyano-3,3-diphenylacrylate (INCI: Octocrylene), 2-ethylhexyl 2-hydroxybenzoate (2-ethylhexyl salicylate, octyl salicylate, INCI: Ethylhexyl Salicylate) and esters of cinnamic acid, preferably 2-ethylhexyl 4-methoxycinnamate (INCI: Ethylhexyl Methoxycinnamate) and isopentyl 4-methoxycinnamate (INCI: Isoamyl p-Methoxycinnamate), 3-(4-(2,2-bis-ethoxycarbonylvinyl)phenoxy)propenyl)methoxysiloxane/dimethylsiloxane copolymer, which is available, for example, under the trade name Parsol® SLX from Hoffmann La Roche.

Preferred inorganic pigments are metal oxides and/or other metal compounds which are insoluble or sparingly soluble in water, in particular oxides of titanium (TiO₂), zinc (ZnO), iron (e.g. Fe₂O₃), zirconium (ZrO₂), silicon (SiO₂), manganese (e.g. MnO), aluminum (Al₂O₃), cerium (e.g. Ce₂O₃), mixed oxides of the corresponding metals, and mixtures of such oxides, and also the sulphate of barium (BaSO₄).

For the purposes of the present invention, the pigments may advantageously also be used in the form of commercially available oily or aqueous predispersions. Dispersion auxiliaries and/or solubility promoters may advantageously be added to these predispersions.

According to the invention, the pigments may advantageously be surface-treated (“coated”), the intention being to form or retain, for example, a hydrophilic, amphiphilic or hydrophobic character. This surface treatment can consist in providing the pigments with a thin hydrophilic and/or hydrophobic inorganic and/or organic coat by methods known per se. For the purposes of the present invention, the various surface coatings may also comprise water.

Inorganic surface coatings for the purposes of the present invention may consist of aluminum oxide (Al₂O₃), aluminum hydroxide Al(OH)₃, or aluminum oxide hydrate (also: alumina, CAS No.: 1333-84-2), sodium hexametaphosphate (NaPO₃)₆, sodium metaphosphate (NaPO₃)ₙ, silicon dioxide (SiO₂) (also: silica, CAS No.: 7631-86-9), or iron oxide (Fe₂O₃). These inorganic surface coatings may be present on their own, in combination and/or in combination with organic coating materials.

Organic surface coatings for the purposes of the present invention may consist of vegetable or animal aluminum stearate, vegetable or animal stearic acid, lauric acid, dimethylpolysiloxane (also: Dimethicone), methylpolysiloxane (Methicone), simethicone (a mixture of dimethylpolysiloxane with an average chain length of from 200 to 350 dimethylsiloxane units and silica gel) or alginic acid. These organic surface coatings may be present on their own, in combination and/or in combination with inorganic coating materials.

Zinc oxide particles and predispersions of zinc oxide particles which are suitable according to the invention are obtainable under the following trade names from the companies listed:

| Trade name | Coating | Manufacturer |
|---|---|---|
| Z-Cote HP1 | 2% Dimethicone | BASF |
| Z-Cote | / | BASF |
| ZnO NDM | 5% Dimethicone | H&R |
| MZ-303S | 3% Methicone | Tayca Corporation |
| MZ-505S | 5% Methicone | Tayca Corporation |

Suitable titanium dioxide particles and predispersions of titanium dioxide particles are available under the following trade names from the companies listed:

| Trade name | Coating | Manufacturer |
|---|---|---|
| MT-100TV | Aluminum hydroxide/stearic | Tayca Corporation |
| | acid | |
| MT-100Z | Aluminum hydroxide/stearic | Tayca Corporation |
| | acid | |
| Eusolex T-2000 | Alumina/Simethicone | Merck KgaA |
| Titanium dioxide | Octyltrimethylsilane | Degussa |
| T805 | | |
| (Uvinul TiO₂) | | |
| Tioveil AQ 10PG | Alumina/Silica | Solaveil/Uniquema |
| Eusolex T-aqua | Water/alumina/sodium | Merck |
| | metaphosphate | |

Further advantageous pigments are latex particles. Latex particles advantageous according to the invention are those described in the following specifications: U.S. Pat. No. 5,663,213 and EP 0 761 201. Particularly advantageous latex particles are those which are formed from water and styrene/acrylate copolymers and are available, for example, under the trade name “Alliance SunSphere” from Rohm & Haas.

Advantageous UV-A filter substances for the purposes of the present invention are dibenzoylmethane derivatives, in particular 4-(tert-butyl)-4′-methoxydibenzoyl-methane (CAS No. 70356-09-1), which is sold by Givaudan under the name Parsole 1789 and by Merck under the trade name Eusolex® 9020.

Advantageous further UV filter substances for the purposes of the present invention are sulfonated, water-soluble UV filters, such as, for example:
phenylene-1,4-bis(2-benzimidazyl)-3,3′-5,5′-tetrasulfonic acid and its salts, particularly the corresponding sodium, potassium or triethanolammonium salts, in particular the phenylene-1,4-bis(2-benzimidazyl)-3,3′-5,5′-tetrasulfonic acid bis-sodium salt with the INCI name Disodium Phenyl Dibenzimidazole Tetrasulfonate (CAS No.: 180898-37-7), which is available, for example, under the trade name Neo Heliopan AP from Haarmann & Reimer;
salts of 2-phenylbenzimidazole-5-sulfonic acid, such as its sodium, potassium or its triethanolammonium salt, and the sulfonic acid itself with the INCI name Phenylbenzimidazole Sulfonic Acid (CAS No. 27503-81-7), which is available, for example, under the trade name Eusolex 232 from Merck, or under Neo Heliopan Hydro from Haarmann & Reimer;
1,4-di(2-oxo-10-sulfo-3-bornylidenemethyl)benzene (also: 3,3′-(1,4-phenylene-dimethylene)bis(7,7-dimethyl-2-oxobicyclo[2.2.1 ]hept-1-ylmethanesulfonic acid) and salts thereof (particularly the corresponding 10-sulfato compounds, in particular the corresponding sodium, potassium or triethanolammonium salt), which is also referred to as benzene-1,4-di(2-oxo-3-bornylidenemethyl-10-sulfonic acid). Benzene-1,4-di(2-oxo-3-bornylidenemethyl-10-sulfonic acid) has the INCI name Terephthalidene Dicamphor Sulfonic Acid (CAS No.: 90457-82-2) and is available, for example, under the trade name Mexoryl SX from Chimex;
sulfonic acid derivatives of 3-benzylidenecamphor, such as, for example, 4-(2-oxo-3-bornylidenemethyl)benzenesulfonic acid, 2-methyl-5-(2-oxo-3-bornylidene-methyl)sulfonic acid and salts thereof.

Further advantageous UV filter substances for the purposes of the present invention are benzoxazole derivatives which are characterized by the following structural formula, 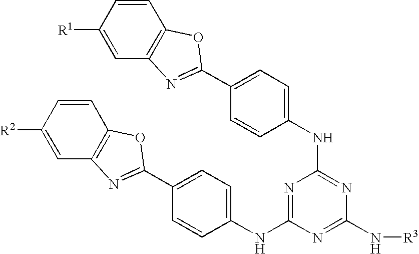
in which R¹, R² and R³, independently of one another, are chosen from the group of branched or unbranched, saturated or unsaturated alkyl radicals having 1 to 10 carbon atoms. It is particularly advantageous according to the invention to choose the radicals R¹ and R² to be the same, in particular from the group of branched alkyl radicals having 3 to 5 carbon atoms. It is also particularly advantageous for the purposes of the present invention if R³ is an unbranched or branched alkyl radical having 8 carbon atoms, in particular the 2-ethylhexyl radical.

A benzoxazole derivative which is particularly preferred according to the invention is 2 ,4-bis[5-1(dimethylpropyl)benzoxazol-2-yl(4-phenyl)imino]-6-(2-ethylhexyl)imino-1,3,5-triazine with the CAS No. 288254-16-0, which is characterized by the structural formula 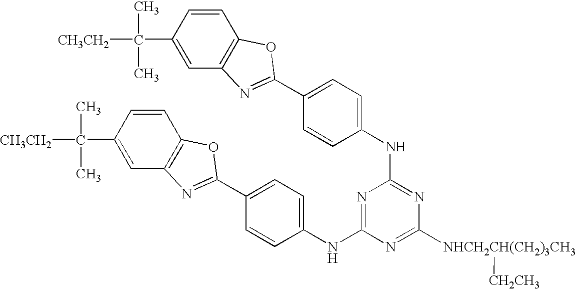
and is available from 3V Sigma under the trade name Uvasorb® K2A.

The benzoxazole derivative or derivatives are advantageously present in the cosmetic preparations according to the invention in dissolved form. In some circumstances, however, it may also be advantageous if the benzoxazole derivative or derivatives are present in pigmentary, i.e. undissolved, form—for example in particle sizes of from 10 nm to 300 nm.

Further advantageous UV filter substances for the purposes of the present invention are so-called hydroxybenzophenones. Hydroxybenzophenones are characterized by the following structural formula: 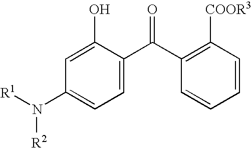
in which
R¹ and R², independently of one another, are hydrogen, C₁-C₂₀-alkyl, C₃-C₁₀-cycloalkyl or C₃-C₁₀-cycloalkenyl, where the substituents R¹ and R², together with the nitrogen atom to which they are bonded, can form a 5-membered or 6-membered ring and
R³ is a C₁-C₂₀-alkyl radical.

A particularly advantageous hydroxybenzophenone for the purposes of the present invention is hexyl 2-(4′-diethylamino-2′-hydroxybenzoyl)benzoate (also: aminobenzophenone), which is characterized by the following structure: 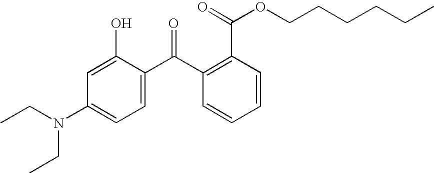
and is available under Uvinul A Plus from BASF.

Advantageous UV filter substances for the purposes of the present invention are also so-called broadband filters, i.e. filter substances which absorb both UV-A and also UV-B radiation.

Advantageous broadband filters or UV-B filter substances are, for example, triazine derivatives, such as, for example,
2,4-bis{[4-(2-ethylhexyloxy)-2-hydroxy]phenyl}-6-(4-methoxyphenyl)-1,3,5-triazine (INCI: Bis-Ethylhexyloxyphenol Methoxyphenyl Triazine), which is available under the trade name Tinosorb® S from CIBA-Chemikalien GmbH;
dioctylbutylamidotriazone (INCI: Diethylhexyl Butamido Triazone), which is available under the trade name UVASORB HEB from Sigma 3V;
Tris(2-ethylhexyl) 4,4′,4″-(1,3,5-triazine-2,4,6-triyltriimino)trisbenzoate, also: 2,4,6-tris[anilino(p-carbo-2′-ethyl-1′-hexyloxy)]-1,3,5-triazine (INCI: Ethylhexyl Triazone), which is sold by BASF Aktiengesellschaft under the trade name UVINUL® T 150;
2-[4,6-bis(2,4-dimethylphenyl)-1,3,5-triazin-2-yl]-5-(octyloxy)phenol (CAS No.: 2725-22-6).

An advantageous broadband filter for the purposes of the present invention is also 2,2′-methylenebis(6-(2H-benzotriazol-2-yl)-4-(1,1,3,3-tetramethylbutyl)phenol), (INCI: Methylene Bis-Benztriazolyl Tetramethylbutylphenol) which is available, for example, under the trade name Tinosorb® M from CIBA-Chemikalien GmbH.

For the purposes of the present invention, an advantageous broadband filter is also 2-(2H-benzotriazol-2-yl)-4-methyl-6-[2-methyl-3-[1,3,3,3-tetramethyl-1-[(trimethylsilyl)oxy]disiloxanyl]propyl]phenol (CAS No.: 155633-54-8) with the INCI name Drometrizole Trisiloxane.

The further UV filter substances may be oil-soluble or water-soluble. Advantageous oil-soluble filter substances are, for example:
3-benzylidenecamphor derivatives, preferably 3-(4-methylbenzylidene)camphor, 3-benzylidenecamphor;
4-aminobenzoic acid derivatives, preferably 2-ethylhexyl 4-(dimethyl-amino)benzoate, amyl 4-(dimethylamino)benzoate;
2,4,6-trianilino(p-carbo-2′-ethyl-1′-hexyloxy)-1,3,5-triazine;
esters of benzalmalonic acid, preferably di(2-ethylhexyl) 4-methoxybenzal-malonate;
esters of cinnamic acid, preferably 2-ethylhexyl 4-methoxycinnamate, isopentyl 4-methoxycinnamate;
derivatives of benzophenone, preferably 2-hydroxy-4-methoxybenzophenone, 2-hydroxy-4-methoxy-4′-methylbenzophenone, 2,2′-dihydroxy-4-methoxy-benzophenone and
UV filters bonded to polymers.

Advantageous water-soluble filter substances are, for example:

Sulfonic acid derivatives of 3-benzylidenecamphor, such as, for example, 4-(2-oxo-3-bornylidenemethyl)benzenesulfonic acid, 2-methyl-5-(2-oxo-3-bornylidene-methyl)sulfonic acid and salts thereof.

A further light protection filter substance to be used advantageously according to the invention is ethylhexyl 2-cyano-3,3-diphenylacrylate (octocrylene), which is available from BASF under the name Uvinul® N 539 T.

Besides the filter substance(s) according to the invention, particularly advantageous preparations for the purposes of the present invention which are characterized by high or very high UV-A protection preferably also comprise further UV-A and/or broadband filters, in particular dibenzoylmethane derivatives [for example 4-(tert-butyl)-4′-methoxydibenzoylmethane] and/or 2,4-bis{[4-(2-ethylhexyloxy)-2-hydroxy]phenyl}-6-(4-methoxyphenyl)-1,3,5-triazine and/or phenylene-1,4-bis(2-benzimidazyl)-3,3′-5,5′-tetrasulfonic acid bis-sodium salt, in each case individually or in any combinations with one another.

The list of given UV filters which can be used for the purposes of the present invention is not of course intended to be limiting.

The preparations according to the invention advantageously comprise the substances which absorb UV radiation in the UV-A and/or UV-B region in a total amount of, for example, from 0.1% by weight to 30% by weight, preferably from 0.5 to 20% by weight, in particular 1.0 to 15.0% by weight, in each case based on the total weight of the preparations, in order to provide cosmetic preparations which protect the hair and/or the skin from the entire range of ultraviolet radiation.

The preparations for the purposes of the present invention can also advantageously comprise further substances which increase the water resistance of the products, in particular when these are to be used as sunscreen products.

For the purposes of the present invention, it is advantageous to use PEG-45 dodecyl glycol copolymer (INCI: PEG-45 Dodecyl Glycol Copolymer [y=z=11 and x=45]) and PEG-22 dodecyl glycol copolymer (INCI: PEG-22 Dodecyl Glycol Copolymer [y=z=4.5 and x=22]) and methoxy PEG-22 dodecyl glycol copolymer (INCI: Methoxy PEG-22 Dodecyl Glycol Copolymer [y=7 and x=22 and R═CH₃]), which are available from AKZO Nobel.

For example, water-soluble or water-dispersible polyoxyethylene-polyoxypropylene block polymers (CTFA name: Polaxamers, CAS No. 9003-11-6) with the following structure are also advantageous: 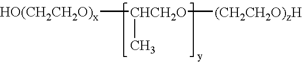
where x, y and z are integers from the range from 2 to 130, in particular from 15 to 100, and x and z are identical, but are chosen independently of y.

Of these, Polaxamer 188 [where x=75, y=30 and z=75), which can be obtained under the trade name Lutrol F 68 (formerly: Pluronic F 68) from BASF, Polaxamer 185 [where x=19, y=30 and z=19] (Lubrajel WA from ISP), Polaxamer 235 [where x=27, y=39 and z=27] (Pluronic F 85 from BASF) and/or Polaxamer 238 [where x=97, y=39 and z=97] (Pluronic F 88 from BASF) are to be used particularly advantageously.

Further advantageous substances which can contribute to the increase in the water resistance, but are incorporated in the oil phase of the preparations according to the present invention, are certain wax components, such as acetylated glycol stearate with tristearin (e.g. Unitwix from ISP with the INCI: Acetylated Glycol Stearate and Tristearin), C₁₈₋₃₆ fatty acid triglyceride (e.g.: Syncrowax HGLC from Crode GmbH with the INCI: C18-36 Acid Triglyceride), and the substances obtainable under the trade names “Performa V 1608” (INCI: C30-38 Olefin/lsopropyl Maleate/MA Copolymer) and “Performa V 825” (synthetic wax) from New Phase Technologies.

It is particularly advantageous for the purposes of the present invention to combine the substances mentioned with one another in order to further improve the water resistance of the preparations.

The examples below are intended to illustrate the present invention without limiting it. The numerical values in the examples are percentages by weight, based on the total weight of the particular preparations.

### EXAMPLES

### 1

Self-Foaming, Foam-Like O/W Lotions and Creams

| | 1 | 2 | 3 | 4 | 5 | 6 | 7 |
|---|---|---|---|---|---|---|---|
| Palmitic acid | | | | 1.00 | | | |
| Stearic acid | 5.00 | 2.00 | 3.00 | | 4.00 | 4.00 | 1.00 |
| Cetylstearyl alcohol | 5.50 | 0.50 | | | | 1.00 | 4.00 |
| Myristyl alcohol | | 1.50 | | | | | 1.00 |
| Cetyl alcohol | | | 8.50 | 2.00 | 2.00 | | |
| PEG-20 stearate | | | 8.50 | | | | 1.00 |
| PEG-30 stearate | 1.00 | | | | 2.00 | | |
| PEG-40 stearate | | | | 5.50 | | | |
| PEG-100 stearate | | 3.00 | | | | 1.00 | |
| Sorbitan monostearate | | | | | 1.50 | | |
| Isopropyl palmitate | | | 2.00 | | | | |
| Cyclomethicone | 6.00 | | | | | | |
| Dimethicone | | | | 4.50 | | 0.50 | 0.50 |
| Caprylic/caprylic | | | | | | | 2.00 |
| triglycerides | | | | | | | |
| Octyl isostearate | | | | | | | 5.00 |
| C12-15 Alkyl benzoates | | | 5.00 | | | | |
| Dicaprylyl carbonate | | | | 2.00 | 5.00 | | |
| Isohexadecane | | | 2.00 | | | | |
| Isoeicosane | 1.0 | | | | | | |
| Mineral oil/paraffin oil | | 5.00 | | 4.50 | | 6.50 | 15.0 |
| Hydrogenated | | 15.0 | | | 5.00 | | |
| polyisobutene | | | | | | | |
| Polydecene | 6.0 | | | | | | |
| Magnesium silicate | 0.10 | | | | | | 0.10 |
| Magnesium aluminum | | 0.20 | | | | 0.10 | |
| silicate | | | | | | | |
| Hectorite | | | | 0.10 | | | |
| Quaternium-18 hectorite | | | | | 0.10 | | |
| Carrageen | 0.10 | | | | 0.10 | | |
| Cellulose gum | | | | | | 0.10 | |
| Xanthan gum | | | | | 0.05 | | |
| Polyacrylic acid | | | | 0.10 | 0.10 | 0.10 | |
| Alkyl acrylates | | | | | | | 0.10 |
| crosspolymers | | | | | | | |
| Hydroxypropyl starch | | 0.25 | 0.50 | | | | |
| phosphate and esters | | | | | | | |
| thereof | | | | | | | |
| Starch or starch | 0.20 | | | | | | |
| derivative (aluminum | | | | | | | |
| starch octenylsuccinate | | | | | | | |
| etc.) | | | | | | | |
| Inorg. pigments (such as | 0.90 | 1.25 | 1.0 | 1.25 | 0.60 | 0.50 | 0.25 |
| boron nitride, zeolites, | | | | | | | |
| kaolin, silicon oxide, talc, | | | | | | | |
| silica dimethyl silylate | | | | | | | |
| etc.) | | | | | | | |
| Citric acid | 0.10 | | | | | | |
| Glycerol | 3.00 | 5.00 | 10.0 | 3.00 | 3.00 | 3.00 | 5.50 |
| Octyl methoxycinnamate | | | | | | | 4.00 |
| Butylmethoxydibenzoylmethane | | | | | | | 3.00 |
| Ethylhexyltriazone | | | | | | | 3.00 |
| Mica | | | | 1.00 | | | |
| iron oxides | | | | 1.00 | | | |
| Titanium dioxide | | | | 4.50 | | | |
| Vitamin A palmitate | | | | 0.10 | 0.20 | | |
| Vitamin E acetate | | | | | 1.00 | 2.00 | |
| BHT | | | | | 0.02 | | 0.02 |
| Disodium EDTA | | | | | 0.10 | | 0.10 |
| Perfume | q.s. | q.s. | q.s. | q.s. | q.s. | q.s. | q.s. |
| Preservative | q.s. | q.s. | q.s. | q.s. | q.s. | q.s. | q.s. |
| Sodium hydroxide | q.s. | q.s. | q.s. | q.s. | | | q.s. |
| Potassium hydroxide | | | | | q.s | q.s | |
| Dyes etc. | q.s | q.s. | q.s | q.s. | q.s | q.s. | q.s |
| Water | ad | ad | ad | ad | ad | ad | ad |
| | 100 | 100 | 100 | 100 | 100 | 100 | 100 |
| pH adjusted to | 6.0-7.5 | 5-6.5 | 6.2-7.0 | 5.0-6.4 | 5.3-6.7 | 6.0-7.5 | 6.0-7.5 |
| Volume % of the base | 60 | 85 | 75 | 40 | 90 | 80 | 95 |
| Volume % oxygen | 40 | 15 | 25 | 60 | 10 | 20 | 5 |

Predispersion of the inorganic gel former and swelling of the hydrocolloid with stirring in the water phase. Combining the fatty phase heated to 75° C. with the water phase heated to 70° C.. Addition of the particulate hydrophobic, hydrophobicized solid substances with stirring. Homogenization by means of a toothed-wheeled dispersing machine (rotor-stator principle) at 65° C.. Stirring for 45 min under reduced pressure and with cooling. Addition of the additives at 30° C. (perfume, active ingredients). Homogenization by means of a toothed-wheel dispersing machine (rotor-stator principle) at 27° C. Gassing of the emulsion at 7-8 bar using a dynamic foam generator (e.g. from HANSA INDUSTRIE MIXER) with oxygen.

### 2

(Foamable O/W Emulsion Make-Up):

| Emulsion | % by wt. |
|---|---|
| Palmitic acid | 2.00 |
| Cetyl alcohol | 2.00 |
| PEG-100 stearate¹ | 2.00 |
| Dimethicone² | 2.50 |
| Paraffin oil³ | 9.50 |
| Dicaprylyl carbonate⁴ | 2.00 |
| Glycerol | 3.00 |
| Mica | 1.00 |
| Iron oxide | 1.00 |
| Titanium dioxide | 4.50 |
| Vitamin A palmitate | 0.10 |
| Sodium hydroxide | q.s. |
| Preservative | q.s. |
| Perfume | q.s. |
| Water, demineralized | ad 100 |

pH adjusted to 6.0-7.5
To produce the foam, 85% by volume of the emulsion IV are foamed with 5% by volume of oxygen and 5% by volume of propane/butane^{a}.
¹Myrj 59p, ICI Surfactants,
²Wacker silicone oil AK 35, Wacker,
³Pionier 6301, DEA mineral oil,
⁴Cetiol CC, Henkel Cognis

### 3

(Foamable O/W Cream)

| Emulsion | % by wt. |
|---|---|
| Stearic acid | 4.00 |
| Cetyl alcohol | 2.00 |
| PEG-30 stearate¹ | 2.00 |
| Sorbitan monostearate² | 1.50 |
| Paraffin oil³ | 5.00 |
| Cyclomethicone⁴ | 5.00 |
| Vitamin E acetate | 1.00 |
| Retinyl palmitate | 0.20 |
| Glycerol | 3.00 |
| Alcohol cosmet. | 10.00 |
| BHT | 0.02 |
| Disodium EDTA | 0.10 |
| Perfume, preservative, dyes | q.s. |
| Potassium hydroxide | q.s. |
| Water | ad 100 |

pH adjusted to 5.0-7.0
To produce the foam, 89% by volume of the emulsion V are foamed with 11% by volume of oxygen^{a}.
¹Myrj 51, ICI Surfactants,
²Glycomol S, Akzo Nobel,
³Pionier 2076, DEA mineral oil
⁴Dow Corning Fluid 245, Dow Corning

### 4

(Foamable O/W Lotion):

| Emulsion | % by wt. |
|---|---|
| Stearic acid | 4.00 |
| Cetylstearyl alcohol | 1.00 |
| PEG-100 stearate¹ | 1.00 |
| Octyldodecanol | 6.50 |
| Dimethicone³ | 2.50 |
| Vitamin E acetate | 2.00 |
| Glycerol | 3.00 |
| Cetyl ricinoleate | 2.00 |
| Perfume, preservative, dyes etc. | q.s. |
| Sodium hydroxide | q.s. |
| Water | ad 100 |

pH adjusted to 6.0-7.5
To produce the foam, 92% by volume of the emulsion VI are foamed with 8% by volume of a propellant gas mixture of propane and butane (4% by volume) and oxygen (4% by volume)^{a}.
¹Myrj 59p, ICI Surfactants,
³Wacker silicone oil AK 50, Wacker

### 5

(Foamable Sunscreen Cream):

| | % by | % | % by | % by |
|---|---|---|---|---|
| | wt. | by vol. | wt. | vol. |
| | Emulsion 11 | | Emulsion 12 | |
| Stearic acid | 5.00 | | 1.00 | |
| Cetyl alcohol | 5.50 | | | |
| Cetylstearyl alcohol | | | 2.00 | |
| PEG-40 stearate | 8.50 | | | |
| PEG-20 stearate | | | 1.00 | |
| Caprylic/capric triglycerides | 4.00 | | 2.00 | |
| C₁₂₋₁₅ Alkyl benzoates | 10.00 | | 15.50 | |
| Cyclomethicone | 4.00 | | | |
| Dimethicone | | | 0.50 | |
| Octyl isostearate | | | 5.00 | |
| Myristyl myristate | | | 2.00 | |
| Ceresin | 1.50 | | | |
| Glycerol | 5.00 | | 10.00 | |
| UVASorb ® K2A | 2.00 | | | |
| Uvinul A Plus ® | 2.00 | | 1.50 | |
| Terephthalidenedicamphorsulfonic | 0.50 | | | |
| acid | | | | |
| Drometrizoletrisiloxane | 1.50 | | | |
| Ethylhexyl methoxycinnamate | 5.00 | | 4.00 | |
| Ethylhexyltriazone | | | 3.00 | |
| Octocrylene | 5.00 | | | |
| Titanium dioxide Uvinul T 805 | 1.00 | | | |
| BHT | | | 0.02 | |
| Na₂H₂EDTA | 0.50 | | 0.10 | |
| Perfume, preservative, | q.s. | | q.s. | |
| Dyes, etc. | q.s. | | q.s. | |
| Potassium hydroxide | q.s. | | q.s. | |
| Water | ad | | ad 100.00 | |
| | 100.00 | | | |
| | pH adjusted | | pH adjusted | |
| | to 6.5-7.5 | | to 5.0-6.0 | |
| Emulsion 1 | | 70 | | |
| Emulsion 2 | | | | 35 |
| Gas (oxygen) | | 30 | | 65 |

To produce the foam, 85% by volume of the emulsion 12, 13 are foamed with oxygen^{a}.

### 6

O/W Emulsions

| | 13 | 14 | 15 | 16 | 17 | 18 | 19 |
|---|---|---|---|---|---|---|---|
| Glycerol monostearate SE | 0.50 | 1.00 | 3.00 | | | 1.50 | |
| Glyceryl stearate citrate | 2.00 | | | 1.00 | 2.00 | | 2.50 |
| Stearic acid | | 3.00 | 0.75 | 2.00 | | | |
| PEG-40 stearate | 0.50 | | | | | 2.00 | |
| PEG-100 stearate | | | 1.50 | | | | |
| Laurylmethicone copolyol | | | | 0.75 | | 0.50 | |
| Cetyl phosphate | | | 0.75 | | 1.00 | | |
| Stearyl alcohol | | | 3.00 | | | 2.00 | 0.50 |
| Cetyl alcohol | 2.50 | 1.00 | | | 0.50 | | 2.00 |
| UVASorb ® K2A | 1.00 | | | 4.00 | | 5.00 | |
| Uvinul ® A Plus | 3.00 | 2.50 | 0.50 | 0.25 | 1.00 | 0.50 | 4.00 |
| Bisethylhexyloxyphenolmethoxyphenyltriazine | | 1.00 | | | | 1.00 | 0.50 |
| Disodiumphenyldibenzimidazoletetrasulfonate | | | | 1.00 | | 2.00 | |
| Ethylhexyltriazone | 2.00 | | | 2.00 | | 2.00 | |
| Diethylhexylbutamidotriazone | | 2.00 | | | | | |
| Ethylhexyl | | 3.50 | | 10.00 | | | |
| methoxycinnamate | | | | | | | |
| Octocrylene | | | | 5.00 | 9.00 | 7.50 | 2.50 |
| Methylenebisbenztriazolyltetramethylbutylphenol | | | 2.00 | | 3.00 | | |
| Ethylhexyl salicylate | | | 3.00 | | | | 5.00 |
| Drometrizole trisiloxane | | | 0.50 | | | 1.00 | |
| Titanium dioxide T 805 | | 1.50 | | | 1.00 | 0.50 | |
| Titanium dioxide MT-100Z | 1.00 | | | 3.00 | 1.00 | | |
| C₁₂₋₁₅ Alkyl benzoates | | 2.50 | | | | 7.00 | 5.00 |
| Dicaprylyl ether | | | 3.50 | | 2.00 | | |
| Butylene glycol dicaprylate/ | 5.00 | | | 5.00 | 3.00 | | |
| dicaprate | | | | | | | |
| Cetearyl isononanoate | | 4.00 | | | | 2.00 | 2.00 |
| Dimethicone | | 0.50 | 1.00 | | 2.00 | | |
| Cyclomethicone | 2.00 | | | 4.50 | | | 0.50 |
| Dimethicone/vinyldimethicone | | 4.00 | | | | | 0.50 |
| crosspolymer | | | | | | | |
| PVP hexadecene copolymer | 0.50 | | | 0.50 | 1.00 | | 1.00 |
| Glycerol | 3.00 | 7.50 | | 7.50 | 5.00 | | 20.00 |
| Xanthan gum | 0.15 | | 0.05 | | | | 0.30 |
| Butylene glycol | 7.00 | 5.00 | | | | 7.00 | |
| Vitamin E acetate | 0.50 | | 0.25 | 0.50 | 0.75 | | 1.00 |
| Dioic acid | 0.25 | | | 0.20 | | 0.25 | |
| Fucogel ® 1000 | | | 1.50 | | | 5.00 | |
| DMDM hydantoin | | 0.60 | 0.40 | 0.20 | | | |
| Methylparaben | 0.15 | | 0.25 | | 0.50 | | |
| Phenoxyethanol | 1.00 | 0.40 | | 0.40 | 0.50 | | 0.60 |
| EDTA | | 0.20 | 0.35 | 0.50 | 0.02 | | 0.03 |
| Ethanol | | 2.00 | 1.50 | | 3.00 | 5.00 | 1.00 |
| Insect repellent 3535 | | | 5.00 | | | | |
| Perfume | 0.20 | 0.20 | | | | 0.30 | 0.40 |
| Water | ad | ad | ad | ad | ad | ad | ad |
| | 100 | 100 | 100 | 100 | 100 | 100 | 100 |
| Emulsion (% by vol.) | 80 | 85 | 50 | 70 | 95 | 60 | 30 |
| Gas oxygen (% by vol.) | 20 | 15 | 50 | 30 | 5 | 40 | 70 |

### 7

Thin-Liquid to Sprayable W/O Emulsions (for Use as Spray or Aerosol)

| | 20 | 21 | 22 | 23 | 24 |
|---|---|---|---|---|---|
| Cetyldimethicone copolyol | 4.00 | | | 2.50 | 3.00 |
| Polyglyceryl-2 | | | 3.00 | | 1.00 |
| dipolyhydroxystearate | | | | | |
| Isostearyl diglyceryl succinate | | | 0.75 | | 0.30 |
| Laurylmethicone copolyol | | | | 2.00 | |
| Polysorbat-65 | | | 2.00 | | 1.50 |
| PEG-100 stearate | | | | 1.20 | 0.70 |
| Cetearyl sulfate | | | 0.25 | | 1.00 |
| Dimethicone | | 4.00 | | | 2.00 |
| Cyclomethicone | 12.00 | 20.00 | | 30.00 | 15.00 |
| UVASorb ® K2A | | | | 0.50 | |
| Uvinul ® A Plus | 3.50 | 2.00 | 0.50 | 4.00 | 0.25 |
| Bisethylhexyloxyphenolmethoxyphenyltriazine | 1.00 | | | 0.50 | |
| Disodiumphenyldibenzimidazoletetrasulfonate | | 1.50 | | | 2.00 |
| Drometrizole trisiloxane | | | | 1.00 | |
| Ethylhexyl methoxycinnamate | 3.00 | 4.00 | | | 10.00 |
| Ethylhexyl salicylate | | | 5.00 | | 3.50 |
| Octocrylene | | 5.00 | | 4.00 | |
| Diethylhexylbutamidotriazone | | 1.00 | | | 6.50 |
| Ethylhexyltriazone | 3.00 | | | | 4.00 |
| Titanium dioxide MT-100 Z | | 0.50 | 1.00 | 1.50 | 0.50 |
| Zinc oxide Z-Cote | 2.00 | | | | 4.00 |
| Dicaprylyl carbonate | 5.00 | | 15.00 | | 4.00 |
| Dihexyl carbonate | | 10.00 | | | |
| C12-15 Alkyl benzoate | 7.00 | | 10.00 | | |
| Mineral oil | 10.00 | | | | 6.00 |
| Cocoglycerides | | 2.00 | | 5.00 | |
| PVP Hexadecene copolymer | | 0.75 | | | 0.40 |
| Glycerol | 5.00 | 12.50 | | 5.00 | 15.50 |
| Sorbitol | 5.00 | | 10.00 | | |
| α-Glucosylrutin | | | | | 0.15 |
| EDTA | | 0.15 | 0.03 | | 0.15 |
| Glycin soya | 0.75 | | | 1.50 | |
| Magnesium sulfate | 0.75 | 1.00 | | 0.45 | 1.00 |
| DMDM hydantoin | | 0.05 | | | 0.10 |
| Phenoxyethanol | 1.00 | 0.75 | 0.50 | | 1.00 |
| Ethanol | 2.00 | | | 5.00 | 1.00 |
| Dye, oil-soluble | 0.02 | | | | |
| Perfume | 0.30 | 0.45 | 0.35 | | 0.15 |
| Water | ad. 100 | ad. 100 | ad. 100 | ad. 100 | ad. 100 |
| Emulsion (% by vol.) | 80 | 85 | 50 | 70 | 95 |
| Gas oxygen (% by vol.) | 20 | 15 | 50 | 30 | 5 |

### 8

W/O Emulsions (Creams & Lotions)

| | 25 | 26 | 27 | 28 | 29 |
|---|---|---|---|---|---|
| Cetyldimethicone copolyol | | | | 4.00 | |
| Polyglyceryl-2 | 5.00 | 4.50 | | | 4.50 |
| dipolyhydroxystearate | | | | | |
| PEG-30 dipolyhydroxystearate | | | 5.00 | 2.00 | |
| UVASorb ® K2A | | | | 2.50 | |
| Uvinul ® A Plus | 3.00 | 1.00 | 0.50 | 0.25 | 2.50 |
| Phenylbenzimidazolesulfonic acid | | 4.00 | | 2.00 | 0.50 |
| Ethylhexyl methoxycinnamate | | 8.00 | | 5.00 | 4.00 |
| Diethylhexylbutamidotriazone | 3.00 | 1.00 | | | 3.00 |
| Ethylhexyltriazone | | | 3.00 | 4.00 | |
| Octocrylene | 7.00 | | 8.00 | | 2.50 |
| Drometrizoletrisiloxane | | | 3.00 | | |
| Titanium dioxide Uvinul ® T | 2.00 | 1.00 | | | |
| 805 | | | | | |
| Titanium dioxide MT-100 TV | | | 3.00 | | 2.00 |
| Zinc oxide Z-Cote ® HP1 | 2.50 | | 6.00 | | |
| Mineral oil | | | 10.0 | | 8.00 |
| Cocoglycerides | 4.00 | 6.50 | | | |
| C12-15 Alkyl benzoates | | | | 9.00 | |
| Dicaprylyl ether | 10.00 | | | | 7.00 |
| Butylene glycol dicaprylate/dicaprate | | | 2.00 | 8.00 | 4.00 |
| Cyclomethicone | 2.00 | | | | 2.00 |
| PVP eicosene copolymer | 0.50 | | | 1.50 | 1.00 |
| Trisodium EDTA | 1.00 | | | 0.35 | |
| Ethylhexyloxyglycerol | | 0.30 | 1.00 | | 0.50 |
| Methylpropanediol | | | | | 7.50 |
| Glycerol | 5.00 | 7.50 | | 7.50 | 2.50 |
| Butylene glycol | | 2.50 | 15.00 | | |
| Glycin soya | | 1.00 | 1.50 | | |
| MgSO₄ | 1.00 | 0.50 | | 0.50 | |
| Vitamin E | 0.50 | | 0.25 | | 1.00 |
| DMDM hydantoin | | 0.60 | | 0.20 | |
| Methylparaben | 0.50 | | | 0.15 | |
| Phenoxyethanol | 0.50 | 0.40 | | 1.00 | 0.60 |
| Dihydroxyacetone | | | | 5.50 | |
| Ethanol | 3.00 | | 4.50 | | 1.00 |
| Perfume | 0.20 | | 0.20 | | 0.20 |
| Water | ad 100 | ad 100 | ad 100 | ad 100 | ad 100 |
| Emulsion (% by vol.) | 80 | 85 | 50 | 70 | 95 |
| Gas oxygen (% by vol.) | 20 | 15 | 50 | 30 | 5 |

### 9

Hydrodispersions (for Use as Lotion or Spray)

| | 30 | 31 | 32 | 33 | 34 |
|---|---|---|---|---|---|
| PEG-40 stearate | | 1.25 | | | |
| Cetyl alcohol | | | | 2.00 | |
| Sodium carbomer | | 0.20 | | 0.30 | |
| Acrylates/C10-30 alkyl acrylate crosspolymer | | | 0.40 | 0.10 | 0.10 |
| Xanthan gum | 0.50 | 0.30 | 0.15 | | 0.50 |
| Dimethicone/vinyldimethicone | | | 5.00 | | 3.00 |
| crosspolymer | | | | | |
| UVASorb ® K2A | | | | 3.50 | |
| Uvinul ® A Plus | 0.25 | 3.50 | 0.50 | 2.00 | 1.50 |
| Bisethylhexyloxyphenolmethoxy | | | 0.25 | | |
| phenyltriazine | | | | | |
| Terephthalidenedicamphor- | | 0.20 | | | 0.50 |
| sulfonic acid | | | | | |
| Disodiumphenyldibenzimidazole | 0.75 | | | | 1.00 |
| tetrasulfonate | | | | | |
| Ethylhexyl methoxycinnamate | | | | 5.00 | 8.00 |
| Methylenebisbenzotriazolyltetra | | 1.00 | | | |
| methylbutylphenol | | | | | |
| Diethylhexylbutamidotriazone | | | 2.00 | | |
| Ethylhexyltriazone | 4.00 | | | 4.00 | |
| Octocrylene | | 4.00 | 10.00 | | 2.50 |
| Titanium dioxide MT-100 Z | 0.50 | | 2.00 | 3.00 | 1.00 |
| C₁₂₋₁₅ Alkyl benzoates | 2.00 | 2.50 | | | |
| Butylene glycol | 4.00 | | | 6.00 | |
| dicaprylate/dicaprate | | | | | |
| Dicaprylyl carbonate | | 3.00 | | | |
| Cyclomethicone | | | 7.50 | | |
| Lanoline | | | | 0.35 | |
| PVP hexadecene copolymer | 0.50 | | | 0.50 | 1.00 |
| Ethylhexyloxyglycerol | | 0.50 | 1.00 | | 0.50 |
| Glycerol | 10.00 | 7.50 | | 5.00 | 15.00 |
| Glycin soya | | 1.50 | 1.00 | | |
| Vitamin E acetate | 0.50 | 0.20 | 0.25 | 0.75 | 1.00 |
| α-Glycosylrutin | | 0.30 | | 0.25 | |
| Trisodium EDTA | | 0.30 | 0.10 | 0.20 | |
| Konkaben LMB ® | 0.20 | | | | 0.15 |
| Methylparaben | 0.50 | | | 0.15 | |
| Phenoxyethanol | 0.50 | | | 1.00 | 0.60 |
| Ethanol | 3.00 | 7.00 | 3.50 | | 1.00 |
| Perfume. dyes | q.s. | q.s. | q.s. | q.s. | q.s. |
| Water | ad 100 | ad 100 | ad 100 | ad 100 | ad 100 |
| Emulsion (% by vol.) | 80 | 85 | 50 | 70 | 95 |
| Gas oxygen (% by vol.) | 20 | 15 | 50 | 30 | 5 |

### 10

Oil Gels

| | 35 | 36 | 37 | 38 | 39 |
|---|---|---|---|---|---|
| Octyldodecanol | 9.00 | 9.00 | | | |
| Caprylic/capric triglyceride | 9.00 | | 6.00 | | |
| C₁₂₋₁5-Alkyl benzoates | | | | 5.00 | 8.00 |
| Butylene glycol dicaprylate/dicaprate | | 9.00 | | | 8.00 |
| Dicaprylyl ether | 9.00 | | | 4.00 | |
| Dicaprylyl carbonate | | 7.00 | | | |
| Ethyl galactomannan (N- | 3.50 | | | | 4.00 |
| Hance ® AG 200) | | | | | |
| C₂₀₋₄₀ fatty | | | | 2.00 | |
| acids + Polyethylene (Performacid ® 350) | | | | | |
| Hydroxyoctacosanylhydroxy- | 2.00 | | | | |
| stearate | | | | | |
| Disteardimonium hectorite | 1.00 | | | | 1.00 |
| Cyclomethicone | | | 15.00 | | 5.00 |
| UVASorb ® K2A | | | | | 1.00 |
| Uvinul ® A Plus | 1.00 | 3.50 | 2.75 | 2.00 | 0.50 |
| Ethylhexyl methoxycinnamate | 6.00 | | | 10.00 | 3.0 |
| Octocrylene | 3.50 | | 7.50 | 10.00 | |
| Ethylhexyl salicylate | | 3.50 | | | 4.00 |
| Ethylhexyltriazone | | | 2.00 | | |
| Diethylhexylbutamidotriazone | | 0.50 | | 3.00 | 4.0 |
| Phenoxyethanol | 0.50 | | | | |
| Perfume, dye | q.s. | q.s. | q.s. | q.s. | q.s. |
| Mineral oil | ad. 100 | ad. 100 | ad. 100 | | |
| Rice oil | | | | ad. 100 | ad. 100 |
| Emulsion (% by vol.) | 80 | 85 | 50 | 70 | 95 |
| Gas oxygen (% by vol.) | 20 | 15 | 50 | 30 | 5 |

### 11

Foams

| | Emulsion | | |
|---|---|---|---|
| | 1 | 2 | 3 |
| Stearic acid | 2 | 2 | |
| Palmitic acid | | | 1.5 |
| Cetyl alcohol | 2.5 | 2 | |
| Stearyl alcohol | | | 3 |
| PEG-100 stearate | | | 3.5 |
| PEG-40 stearate | | 2 | |
| PEG-20 stearate | 3 | | |
| Sorbitan stearate | | 0.8 | |
| C₁₂₋₁₅ Alkyl benzoate | 5 | | |
| C₁₂₋₁₃ Alkyl tartrate | | | 7 |
| Butylene glycol | | 6 | |
| dicaprylate/dicaprate | | | |
| Dicaprylyl ether | | | 2 |
| Cyclomethicone | | 2 | 3 |
| Butylene glycol | 1 | | |
| Isohexadecane | 2 | | |
| Methylpropanediol | | | |
| Propylene glycol | | | 5 |
| Glycerol | 5 | 7 | |
| UVASorb ® K2A | | | 2 |
| Uvinul A Plus ® | 2 | | |
| NeoHeliopan ® AP | | | |
| Phenylbenzimidazolesulfonic | | | |
| acid | | | |
| Ethylhexylmethoxycinnamate | | | |
| Ethylhexyltriazone | 2 | 2 | 2 |
| Octocrylene | 2 | | |
| Bisethylhexyloxyphenol- | | 3 | 3 |
| methoxyphenyltriazine | | | |
| Glycyrrhiza Inflata | 0.05 | | |
| Creatine | | | 1 |
| Creatinine | | | 0.1 |
| Vitamin E acetate | | 0.5 | |
| BHT | | | 0.1 |
| Na₂H₂EDTA | 0.50 | | |
| Perfume, preservative | q.s. | q.s. | q.s. |
| Dyes, etc. | q.s. | q.s. | q.s. |
| Sodium hydroxide | q.s. | | q.s. |
| Potassium hydroxide | | q.s. | |
| Water | ad 100.0 | ad 100.0 | ad 100.0 |

### 12

| | Emulsion | | | |
|---|---|---|---|---|
| | 4 | 5 | 6 | 7 |
| Stearic acid | 1.5 | | | |
| Palmitic acid | | | 3 | 3 |
| Cetyl alcohol | | 3 | | |
| Cetylstearyl alcohol | | | 2 | 2 |
| Stearyl alcohol | 3 | | | |
| PEG-100 stearate | | 4 | | |
| PEG-40 stearate | 3 | | | |
| PEG-20 stearate | | | 3 | 3 |
| Sorbitan stearate | 1 | | | |
| Tridecyl trimellitate | | 5 | | |
| C₁₂₋₁₅ Alkyl benzoate | | | 3 | 3 |
| Butylene glycol | 8 | | | |
| dicaprylate/dicaprate | | | | |
| Octyldodecanol | | 2 | | |
| Cocoglycerides | | | | 2 |
| Dicaprylyl ether | | | 2 | 2 |
| Cyclomethicone | | | | |
| Dimethicone | 1 | | 2 | 2 |
| Isohexadecane | | 3 | | |
| Methylpropanediol | | 4 | | |
| Propylene glycol | | | | |
| Glycerol | 5 | | 6 | 6 |
| NeoHeliopan ® AP | | 2 | | |
| Phenylbenzimidazole- | 1 | 4 | 1 | 1 |
| sulfonic acid | | | | |
| Ethylhexyl | 5 | | 4 | 4 |
| methoxycinnamate | | | | |
| Ethylhexyltriazone | | | | |
| Diethyl- | 1 | | | |
| hexylbutamidotriazone | | | | |
| Butylmethoxydibenzoyl- | 2.5 | | 2 | 2 |
| methane | | | | |
| Bisethyl- | 2 | | | |
| hexyloxyphenol- | | | | |
| methoxyphenyltriazine | | | | |
| Licochalcone A | | | | 0.01 |
| Taurine | | 0.3 | | |
| Vitamin E acetate | 0.2 | | 0.3 | 0.3 |
| BHT | | 0.05 | | |
| Na₂H₂EDTA | | | 0.4 | 0.4 |
| Perfume, preservative | q.s. | q.s. | q.s. | q.s. |
| Dyes, etc. | q.s. | q.s. | q.s. | q.s. |
| Sodium hydroxide | q.s. | q.s. | q.s. | q.s. |
| Potassium hydroxide | | | | q.s. |
| Water | ad 100.0 | ad 100.0 | ad 100.0 | ad 100.0 |

To produce the foam, 80-97% by volume of the emulsion I are foamed with 3-20% by volume of a suitable gas (e.g. propane/butane, compressed air, nitrogen).

### 13

| Gel | 1 | 2 | 3 | 4 |
|---|---|---|---|---|
| Acrylate copolymer | | | 5.00 | |
| NeoHeliopan ® AP | | 1.50 | | |
| Phenylbenzimidazolesulfonic acid | 1.50 | 2.00 | | 2.00 |
| Uvinul A Plus ® | | | | 1.00 |
| Unispheres UEL-611 ® | 0.50 | | 0.15 | |
| Carbomer | | 0.90 | | 0.90 |
| Acrylate/C10-30 alkyl acrylate | | 0.80 | | 0.80 |
| crosspolymer | | | | |
| Butylene glycol | | | 3.00 | |
| Glycerol | 4.00 | 4.00 | | 4.00 |
| Dihydroxyacetone | | | 3.00 | |
| Alcohol cosmet. | 8.00 | 12.00 | 5.00 | 12.00 |
| Disodium EDTA | 1.00 | 1 | | 1 |
| Perfume, preservative, dyes | q.s. | q.s. | q.s. | q.s. |
| Sodium hydroxide | q.s. | q.s. | q.s. | q.s. |
| Water | ad 100 | ad 100 | ad 100 | ad 100 |

1.-8. (canceled)

9. A method for the prophylaxis or treatment of defects of skin or cutaneous appendages, wherein the method comprises applying to the skin or the cutaneous appendages a cosmetic or dermatological preparation which comprises oxygen.

10. The method of claim 9, wherein the defects are caused by at least one of extrinsic disturbances, intrinsic disturbances, age-related disturbances and UV-induced disturbances.

11. The method of claim 9, wherein the defects of the skin manifest themselves in at least one of lines, wrinkles, slackening of skin, slackening of tissue, disorders in regeneration of the skin, circulation disorders of the skin, age spots, sensitive skin, itching, stress sensitivity of the skin, inflamed and sensitive skin conditions, dry skin conditions, atopic eczema, psoriasis, redness, rashes, blisters, pigment disorders, uneven skin tone and sunburn.

12. The method of claim 9, wherein the defects of the cutaneous appendages manifest themselves in at least one of disorders in regeneration of the cutaneous appendages, chapping, hair loss, deformation, splitting, discoloration and growth inhibition.

13. The method of claim 9, wherein the cosmetic or dermatological preparation comprises molecular oxygen.

14. The method of claim 13, wherein the cosmetic or dermatological preparation comprises oxygen in a concentration of from 1% to 90% by volume, based on a total volume of the preparation.

15. The method of claim 9, wherein the cosmetic or dermatological preparation is a foam formulation which comprises from 0.5% to 90% by volume of molecular oxygen, based on a total volume of the formulation.

16. The method of claim 15, wherein the foam formulation comprises from 5% to 50% by volume of molecular oxygen.

17. The method of claim 16, wherein the foam formulation comprises from 10% to 40% by volume of molecular oxygen.

18. A method for the prophylaxis or treatment of defects of skin or cutaneous appendages, wherein the method comprises applying to at least one of skin and scalp a cosmetic or dermatological preparation which comprises an emulsifier system of
(a) at least one emulsifier A selected from unsaturated fatty acids having a chain length of from 10 to 40 carbon atoms,
(b) at least one emulsifier B selected from polyethoxylated fatty acid esters having a chain length of from 10 to 40 carbon atoms and a degree of ethoxylation of from 5 to 100, and
(c) at least one coemulsifier C selected from fatty alcohols having a chain length of from 10 to 40 carbon atoms,
and from 1% to 90% by volume, based on a total volume of the preparation, of molecular oxygen.

19. The method of claim 18, wherein the preparation is applied at least once daily.

20. The method of claim 18, wherein emulsifiers A, B and C are present in a total concentration of from 2% to 20% by weight, based on a total weight of the preparation.

21. The method of claim 20, wherein emulsifiers A, B and C are present in a total concentration of from 5% to 15% by weight.

22. The method of claim 21, wherein emulsifiers A, B and C are present in a total concentration of from 7% to 13% by weight.

23. The method of claim 18, wherein the defects are caused by at least one of extrinsic disturbances, intrinsic disturbances, age-related disturbances and UV-induced disturbances.

24. A method for the prophylaxis or treatment of defects of skin or cutaneous appendages, wherein the method comprises applying to at least one of skin and scalp a cosmetic or dermatological preparation which comprises from 0.1% to 8% by weight, based on a total weight of the preparation, of one or more substances selected from hydrocolloids and gel formers, and from 1% to 90% by volume, based on a total volume of the preparation, of molecular oxygen.

25. The method of claim 24, wherein the preparation is applied at least once daily.

26. The method of claim 24, wherein the defects are caused by at least one of extrinsic disturbances, intrinsic disturbances, age-related disturbances and UV-induced disturbances.

27. The method of claim 24, wherein the preparation comprises the one or more substances in a concentration of from 0.2% to 6% by weight.

28. The method of claim 27, wherein the preparation comprises the one or more substances in a concentration of from 0.3% to 4% by weight.
